(19) United States
(12) Patent Application Publication
(10) Pub. No.: US 2018/0258100 A1
(43) Pub. Date: Sep. 13, 2018

(54) RAPAMYCIN ANALOGS SHOWING IMPROVED mTORC1 SPECIFICITY

(71) Applicants: Matthew Alan GREGORY, Cambridge (GB); Steven MOSS, Cambridge (GB); Michael VELARDE, Novato, CA (US); Timothy POWERS, Novato, CA (US); Stelios TZANNIS, Corte Madera, CA (US)

(72) Inventors: Matthew Alan GREGORY, Cambridge (GB); Steven MOSS, Cambridge (GB); Michael VELARDE, Novato, CA (US); Timothy POWERS, Novato, CA (US); Stelios TZANNIS, Corte Madera, CA (US)

(21) Appl. No.: 15/756,034

(22) PCT Filed: Aug. 26, 2016
(86) PCT No.: PCT/US2016/049124

§ 371 Date: Feb. 27, 2018

Related U.S. Application Data
(60) Provisional application No. 62/211,567, filed on Aug. 28, 2015.

(51) Int. Cl. C07D 498/18; A61P 25/28; A61P 25/08; A61P 35/02; A61P 37/02

(52) U.S. Cl. CPC C07D 498/18; A61P 25/28; A61P 25/08; A61K 38/13; A61P 37/02; A61K 2300/00; A61P 35/02

(57) ABSTRACT

In various embodiments novel rapamycin analogs are provides that show improved mTORC1 specificity.

### CROSS-REFERENCE TO RELATED APPLICATIONS

This application claims benefit of and priority to U.S. Ser. No. 62/211,567, filed on Aug. 28, 2015, which is incorporated herein by reference in its entirety for all purposes.

### STATEMENT OF GOVERNMENTAL SUPPORT

[Not Applicable]

### BACKGROUND

Rapamycin (sirolimus) (FIG. 1) is a lipophilic macrolide produced by Streptomyces hygroscopicus NRRL 5491 (Sehgal et al. (1975) J. Antibiotics, 28: 727-733; Vzina et al. (1975) J. Antibiotics, 28: 721-726; U.S. Pat. No. 3,929,992; U.S. Pat. No. 3,993,749, etc.) with a 1,2,3-tricarbonyl moiety linked to a pipecolic acid lactone (Paiva et al. (1991) J. Nat. Prod. 54: 167-177).

Rapamycin has demonstrated pharmacological utility in a large number of contexts. For example, rapamycin shows antifungal activity, against Candida species and also against filamentous fungi (Baker et al., 1978; Sehgal et al., 1975; Vezina et al., 1975; U.S. Pat. No. 3,929,992; U.S. Pat. No. 3,993,749). Rapamycin also inhibits cell proliferation by targeting signal transduction pathways in a variety of cell types. Thus, for example, in T cells rapamycin inhibits signaling from the IL-2 receptor and subsequent autoproliferation of the T cells resulting in immunosuppression. The inhibitory effects of rapamycin are not limited to T cells, since rapamycin inhibits the proliferation of many mammalian cell types (Brunn et al, 1996). Rapamycin is, therefore, a potent immunosuppressant with established or predicted therapeutic applications in the prevention of organ allograft rejection and in the treatment of autoimmune diseases (Kahan et at, 1991), and the like.

Rapamycin is also believed to find utility in the treatment of various cancers. Rapamycin has also shown value in the treatment of chronic plaque psoriasis (Kirby and Griffiths (2001) Br. J. Dermatol., 144: 37-43.), the potential use of effects such as the stimulation of neurite outgrowth in PC12 cells (Lyons et al. (1994) Proc. Natl. Acad. Sci. USA, 91: 3191-3195), the block of the proliferative responses to cytokines by vascular and smooth muscle cells after mechanical injury (Gregory et al. (1993) Transplantation, 55(6): 1409-1418) and its role in prevention of allograft fibrosis (Waller and Nicholson (2001) Br. J. Surg. 88: 1429-1441) are areas of intense research (Kahan and Camardo (2001) Transplantation, 72: 1181-1193). Recent reports reveal that rapamycin is associated with lower incidence of cancer in organ allograft patients on long-term immunosuppressive therapy than those on other immunosuppressive regimes, and that this reduced cancer incidence is due to inhibition of angiogenesis (Guba et al. (2002) Nat. Med. 8: 128-135).

Rapamycin has also found utility in the treatment of lupus. Lupus is a multisystem autoimmune disease where many organs, including the kidney, can be affected. It is a chronic inflammatory disease the pathophysiology of which is manifested by the production of autoantibodies directed against multiple self-antigens, particularly those of nuclear origin. This dysregulation of the immune system results in a loss of self-tolerance, and is mediated by both T and B cells. (Reddy et al. (2008) Arthritis Res. & Therap., 2008, 10: R127 and references therein).

There are very few medications approved for the treatment of lupus (Francis and Peri (2009) Expert Opin. Pharamacotherapy, 10:1481-1494; Mok (2010) Expert Opin. Emerg. Drugs, 15: 53-70). These include: Prednisone (flare up and maintenance treatment), hydroxychloroquine (discoid lupus and SLE), aspirin (arthritis and pleurisy), triamcinolone hexacetonide (discoid lupus), and most recently Benlysta (SLE). In addition, several other agents are regularly prescribed including azathioprine (as a corticosteroid sparing agent), and in more aggressive regimens corticosteroids in combination with variations of 20 cyclophosphamide, mycophenolate mofetil, or the calcineurin inhibitors such as cyclosporine and tacrolimus (Mok (2010) Expert Opin. Emerg. Drugs, 15: 53-70). For patients who are intolerant or refractory to the above listed agents, several biological agents have been utilized including intravenous immunoglobulin and the B cell depleting agent rituximab, although safety concerns have been raised about the latter through a potential link to progressive multifocal leukoencephalopathy infection.

It has been shown that mTOR (mammalian target of rapamycin) activity is upregulated in the T cells of autoimmune patients including lupus and multiple sclerosis (MS) (Fernandez et al. (2009) J. Immunol., 182: 2063-2073), and that inhibition of mTORC1 by rapamycin and its analogs inhibits antigen-induced IL-2 driven T and B cell proliferation. Moreover, the activity of rapamycin and its analogues do not block proliferation of all T cell subtypes, and actually induce selective expansion of regulatory T cells (Tregs) which are important in maintaining immune selftolerance (Donia et al. (2009) J. Autoimmun. 33: 135-140; Esposito et al. (2010)J. Neuroimmunol., 220: 52-63).

Abnormal T cell activation in SLE is linked to sustained elevation of the mitochondrial transmembrane potential, which is in turn controlled by a series of metabolic and stress related inputs (Peri et al. (2004) Trends Immunol. 25: 360-367; Fernandez, et al. (2009) J. Immunol., 182: 2063-2073; Fernandez and Peri (2009) Autoimmun. Rev., 8: 184-189). mTOR is a sensor for these inputs and as a consequence elevated mTOR signaling is observed in lupus T cells, an effect which is normalized by treatment with rapamycin (Peri et al. (2004) supra.; Fernandez, et al. (2009) supra.). Moreover, two independent studies have identified a network of genes that are dysregulated in lupus/nephritis associated disease. There is a strong correlation between the abnormal transcription of these gene networks and mTOR signaling, and treatment with rapamycin returns the levels of gene transcription to asymptomatic levels. (Reddy et al. (2008) Arthritis Res. Ther. 10:R127; Wu et al. (2007) J. Clin. Invest. 117: 2186-2196).

The kinase mTOR (mammalian target of rapamycin) is part of a master regulatory pathway of cell metabolism involving nutrient, growth factor and stress responses (Laplante and Sabatini (2012) Cell, 149(2): 274-293).

Rapamycin, an FDA approved compound, inhibits mTOR signaling, leading to extension of lifespan in a number of species (Harrison et al. (2009) Nature, 460(7253): 392-395), yet it can induce adverse effects (Lamming et al. (2012) Science, 335(6076): 1638-1643). Rapamycin is believed to inhibit mTORC1 directly and mTORC2 indirectly upon chronic treatment (Sarbassov et al. (2006) Mol. Cell, 22(2): 159-68). Recent evidence has revealed that inhibition of mTORC1 is responsible for effects related to lifespan extension, while inhibition of mTORC2 is uncoupled from longevity and is responsible for several of the adverse effects of rapamycin, such as impaired insulin sensitivity, glucose homeostasis, and lipid dysregulation (Lamming et al. (2012) supra).

As noted above, the therapeutic potential of rapamycin has been established in many chronic diseases, from Alzheimer's and Parkinson's disease to diabetes and cardiovascular disease (King et al. (2008) Mol. Pharmacol. 73(4): 1052-1063; Flynn et al. (2013) Aging Cell, 12(5): 851-662). However, the prohibitive safety profile of rapamycin for chronic treatment has limited its use for the treatment of various diseases.

### SUMMARY

Various embodiments contemplated herein may include, but need not be limited to, one or more of the following:

### Embodiment 1

A compound of formula (I):

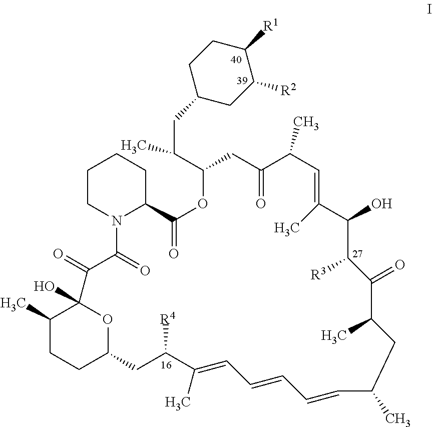

or a pharmaceutically acceptable salt thereof, wherein: R¹ is OH or OCH₃ R² is H or F R³ is H, OH, or OCH₃; and R⁴ is OH or OCH₃.

### Embodiment 2

The compound of embodiment 1, wherein said compound is in pure chiral form as a single diastereomer of formula II:

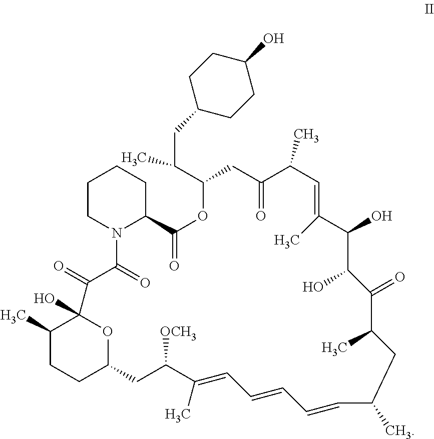 [SMILES: CO[C@H]1C[C@@H]2CC[C@@H](C)[C@@](O)(O2)C(=O)C(=O)N2CCCC[C@H]2C(=O)O[C@H]([C@H](C)C[C@H]2CC[C@H](O)CC2)CC(=O)[C@H](C)/C=C(\C)[C@@H](O)[C@@H](O)C(=O)[C@H](C)C[C@H](C)/C=C/C=C/C=C/1C]

### Embodiment 3

The compound of embodiment 1, wherein said compound is in pure chiral form as a single diastereomer of formula III:

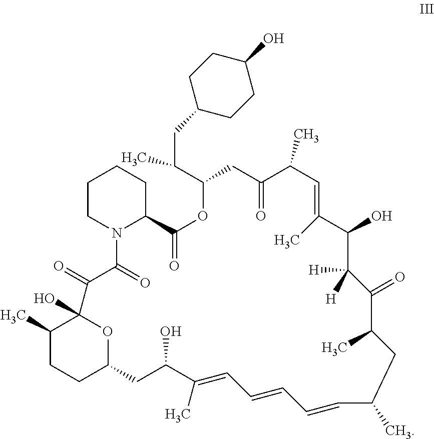

### Embodiment 4

The compound of embodiment 1, wherein said compound is in substantially pure chiral form as a single diastereomer of formula IV:

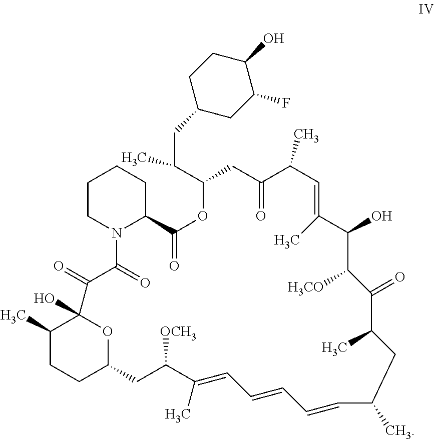 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](F)C2)CC(=O)[C@H](C)/C=C(\C)[C@@H](O)[C@@H](OC)C(=O)[C@H](C)C[C@H](C)/C=C/C=C/C=C/1C]

### Embodiment 5

The compound of embodiment 1, wherein said compound is in substantially pure chiral form as a single diastereomer of formula V:

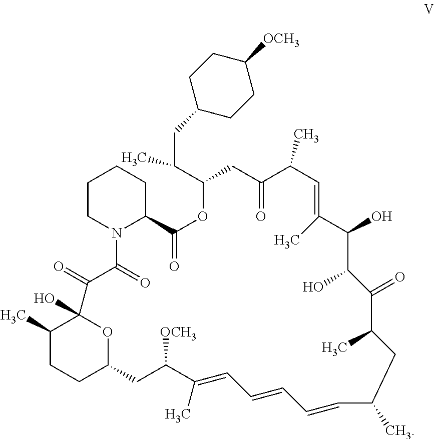 [SMILES: CO[C@H]1C[C@@H]2CC[C@@H](C)[C@@](O)(O2)C(=O)C(=O)N2CCCC[C@H]2C(=O)O[C@H]([C@H](C)C[C@H]2CC[C@H](OC)CC2)CC(=O)[C@H](C)/C=C(\C)[C@@H](O)[C@@H](O)C(=O)[C@H](C)C[C@H](C)/C=C/C=C/C=C/1C]

### Embodiment 6

The compound of embodiment 1, wherein said compound is in substantially pure chiral form as a single diastereomer of formula VI:

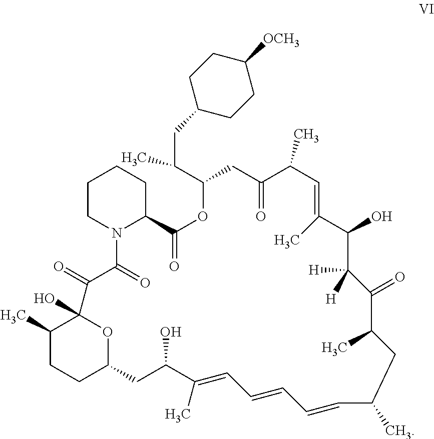

### Embodiment 7

The compound according to any one of embodiments 1-6, wherein said compound is a preferential mTORC1 inhibitor.

### Embodiment 8

A pharmaceutical formulation comprising:
compound according to any one of embodiments 1-7; and
a pharmaceutically acceptable carrier or excipient.

### Embodiment 9

The formulation of embodiment 8, wherein said formulation is a unit dosage formulation.

### Embodiment 10

The formulation according to any one of embodiments 8-9, wherein said formulation is sterile.

### Embodiment 11

The formulation according to any one of embodiments 8-10, wherein said formulation is formulated for administration via a route selected from the group consisting of administration via inhalation, aerosol administration, intravenous administration, intraarterial administration, oral administration, parenteral delivery, rectal administration, subdural administration, systemic administration, topical administration, transdermal delivery, and vaginal administration.

### Embodiment 12

A compound according to any one of embodiments 1-7, or a pharmaceutical formulation according to any one of embodiments 8-11 for use in one or more of the following: the treatment of a tauopathy, the treatment of an mTORopathy (such as tuberous sclerosis complex (TSC), focal cortical dysplasia (FCD), ganglioglioma, hemimegalencephaly, neurofibromatosis 1, Sturge-Weber syndrome, Cowden syndrome, PMSE (Polyhydramnios, Megalencephaly, Symptomatic Epilepsy)), the treatment of an mTORopathy associated with epileptic seizures, the treatment of familial multiple discoid fibromas (FMDF), the treatment of an epilepsy/epileptic seizures (both genetic and acquired forms of the disease such as familial focal epilepsies, epileptic spasms, infantile spasms (IS), status epilepticus (SE), temporal lobe epilepsy (PLE) and absence epilepsy), the treatment of rare diseases associated with a dysfunction of mTORC1 activity (e.g., lymphangioleiomyomatosis (LAM), Leigh's syndrome, Friedrich's ataxia, Diamond-Blackfan anemia, etc.), the treatment of the treatment of metabolic diseases (e.g., obesity, Type II diabetes, etc.), the treatment of autoimmune and inflammatory diseases (e.g., Systemic Lupus Erythematosus (SLE), multiple sclerosis (MS) psoriasis, etc.), the treatment of cancer, the treatment of a fungal infection, the treatment of a proliferative disease, the maintenance of immunosuppression, the treatment of transplant rejection, the treatment of traumatic brain injury, the treatment of autism, the treatment of lysosomal storage diseases and the treatment of neurodegenerative diseases associated with an mTORC1 hyperactivity (e.g., Parkinson's, Huntington's disease, etc.) and generally treatment of disorders that result in hyperactivation of the mTORC1 pathway.

### Embodiment 13

The compound or pharmaceutical formulation of embodiment 12, for use in the treatment of a tauopathy.

### Embodiment 14

The compound or pharmaceutical formulation of embodiment 13, for use in the treatment of a tauopathy selected from the group consisting of progressive supranuclear palsy, dementia pugilistica (chronic traumatic encephalopathy), frontotemporal dementia, lytico-bodig disease (parkinson-dementia complex of guam), tangle-predominant dementia (with nfts similar to ad, but without plaques), ganglioglioma and gangliocytoma, meningioangiomatosis, subacute sclerosing panencephalitis, lead encephalopathy, tuberous sclerosis, Pick's disease, corticobasal degeneration (tau proteins are deposited in the form of inclusion bodies within swollen or “ballooned” neurons), Alzheimer's disease, Parkinson's disease, Huntington's disease, frontotemporal dementia, frontotemporal lobar degeneration.

### Embodiment 15

The compound or pharmaceutical formulation of embodiment 12, for use in the treatment of an mTORpathy.

### Embodiment 16

The compound or pharmaceutical formulation of embodiment 15, wherein said mTORpathy comprises a pathology selected from the group consisting tuberous sclerosis complex (TSC), focal cortical dysplasia (FCD), ganglioglioma, hemimegalencephaly, neurofibromatosis 1, Sturge-Weber syndrome, Cowden syndrome, and PMSE (Polyhydramnios, Megalencephaly, Symptomatic Epilepsy)).

### Embodiment 17

The compound or pharmaceutical formulation of embodiment 12, for use in the treatment of a pathology selected from the group consisting of epilepsy, neurodegeneration, rare and genetic disease with mTORC1 hyperactivity, metabolic disease, and traumatic brain injury.

### Embodiment 18

The compound or pharmaceutical formulation of embodiment 12, for use in the treatment of cancer.

### Embodiment 19

The compound or pharmaceutical formulation of embodiment 18, wherein said cancer is a cancer selected from the group consisting of acute lymphoblastic leukemia (ALL), acute myeloid leukemia (AML), Adrenocortical carcinoma, kaposi sarcoma, anal cancer, appendix cancer, astrocytomas, atypical teratoid/rhabdoid tumor, bile duct cancer, extrahepatic cancer, bladder cancer, bone cancer, brain stem glioma, astrocytomas, spinal cord tumors, central nervous system atypical teratoid/rhabdoid tumor, central nervous system embryonal tumors, central nervous system germ cell tumors, craniopharyngioma, ependymoma, breast cancer, bronchial tumors, burkitt lymphoma, carcinoid tumors, cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, bile duct cancer, extrahepatic cancer, ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, intraocular melanoma, retinoblastoma, fibrous histiocytoma of bone, malignant, and osteosarcoma, gallbladder cancer, gastric (stomach) cancer, gastrointestinal carcinoid tumor, gastrointestinal stromal tumors (GIST), ovarian cancer, testicular cancer, extracranial cancers, extragonadal cancers, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kidney cancer, langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer, lymphoma, cutaneous T-Cell cancer, Hodgkin, non-Hodgkin, primary central nervous system (CNS)), macroglobulinemia, Waldenström, male breast cancer, melanoma, merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma, skin cancer, small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

### Embodiment 20

The compound or pharmaceutical formulation of embodiment 18, wherein said cancer is a cancer selected from the group consisting of brain cancer, breast cancer, central nervous system cancer, cervical cancer, colorectal cancer, testicular cancer, ovarian cancer, leukemia, a lymphoma, a melanoma, a soft tissue sarcoma, testicular cancer, and thyroid cancer.

### Embodiment 21

The compound or pharmaceutical formulation of embodiment 12, for use in the prevention of transplant rejection.

### Embodiment 22

The compound or pharmaceutical formulation of embodiment 21, for use in combination with a calcineurin inhibitor and/or glucocorticoid for the prevention of transplant rejection.

### Embodiment 23

The compound or pharmaceutical formulation of embodiment 21, for use in combination with cyclosporine for the prevention of transplant rejection.

### Embodiment 24

The compound or pharmaceutical formulation of embodiment 12, for use in the treatment of an autoimmune disease.

### Embodiment 25

The compound or pharmaceutical formulation of embodiment 24, wherein said autoimmune disease comprises lupus.

### Embodiment 26

The compound or pharmaceutical formulation of embodiment 24, wherein said autoimmune disease comprises multiple sclerosis.

### Embodiment 27

The compound or pharmaceutical formulation of embodiment 12, for use in the treatment of an infection, autism, or a lysosomal storage disease.

### Embodiment 28

A method of treating a mammal for a pathology/condition selected from the group consisting a tauopathy, an mTORopathy (e.g., such as tuberous sclerosis complex (TSC), focal cortical dysplasia (FCD), ganglioglioma, hemimegalencephaly, neurofibromatosis 1, Sturge-Weber syndrome, Cowden syndrome, PMSE (Polyhydramnios, Megalencephaly, Symptomatic Epilepsy)), an mTORopathy associated with epileptic seizures, familial multiple discoid fibromas (FMDF), epilepsy/epileptic seizures (both genetic and acquired forms of the disease such as familial focal epilepsies, epileptic spasms, infantile spasms (IS), status epilepticus (SE), temporal lobe epilepsy (PLE) and absence epilepsy), rare diseases associated with a dysfunction of mTORC1 activity (e.g., such as lymphangioleiomyomatosis (LAM), Leigh's syndrome, Friedrich's ataxia, Diamond-Blackfan anemia, etc.), metabolic diseases (e.g., such as obesity, Type II diabetes, etc.), autoimmune and inflammatory diseases (e.g., such as Systemic Lupus Erythematosus (SLE), multiple sclerosis (MS) psoriasis, etc.), cancer, a fungal infection, a proliferative disease, the maintenance of immunosuppression, the treatment of transplant rejection, a traumatic brain injury, autism, a lysosomal storage disease, a neurodegenerative diseases associated with mTORC1 hyperactivity (e.g., such as Parkinson's, Huntington's disease, etc.), and disorders that result in hyperactivation of the mTORC1 pathway, in a mammal, said method comprising administering said mammal an effective amount of a compound according to any one of embodiments 1-7, or a pharmaceutical formulation according to any one of embodiments 8-11.

### Embodiment 29

The method of embodiment 28, wherein said pathology comprises a tauopathy.

### Embodiment 30

The method of embodiment 29, wherein said pathology comprises a tauopathy selected from the group consisting of progressive supranuclear palsy, dementia pugilistica (chronic traumatic encephalopathy), frontotemporal dementia, lytico-bodig disease (parkinson-dementia complex of guam), tangle-predominant dementia (with nfts similar to ad, but without plaques), ganglioglioma and gangliocytoma, meningioangiomatosis, subacute sclerosing panencephalitis, lead encephalopathy, tuberous sclerosis, Pick's disease, corticobasal degeneration (tau proteins are deposited in the form of inclusion bodies within swollen or “ballooned” neurons), Alzheimer's disease, Parkinson's disease, Huntington's disease, frontotemporal dementia, frontotemporal lobar degeneration.

### Embodiment 31

The method of embodiment 28, wherein said pathology comprises an mTORpathy.

### Embodiment 32

The method of embodiment 31, wherein said mTORpathy comprises a pathology selected from the group consisting tuberous sclerosis complex (TSC), focal cortical dysplasia (FCD), ganglioglioma, hemimegalencephaly, neurofibromatosis 1, Sturge-Weber syndrome, Cowden syndrome, and PMSE (Polyhydramnios, Megalencephaly, Symptomatic Epilepsy)).

### Embodiment 33

The method of embodiment 28, wherein said pathology comprises a pathology selected from the group consisting of epilepsy, neurodegeneration, rare and genetic disease with mTORC1 hyperactivity, metabolic disease, and traumatic brain injury.

### Embodiment 34

The method of embodiment 28, wherein said pathology comprises a cancer.

### Embodiment 35

The method of embodiment 34, wherein said pathology comprises a cancer selected from the group consisting of acute lymphoblastic leukemia (ALL), acute myeloid leukemia (AML), Adrenocortical carcinoma, kaposi sarcoma, anal cancer, appendix cancer, astrocytomas, atypical teratoid/rhabdoid tumor, bile duct cancer, extrahepatic cancer, bladder cancer, bone cancer, brain stem glioma, astrocytomas, spinal cord tumors, central nervous system atypical teratoid/rhabdoid tumor, central nervous system embryonal tumors, central nervous system germ cell tumors, craniopharyngioma, ependymoma, breast cancer, bronchial tumors, burkitt lymphoma, carcinoid tumors, cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, bile duct cancer, extrahepatic cancer, ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, intraocular melanoma, retinoblastoma, fibrous histiocytoma of bone, malignant, and osteosarcoma, gallbladder cancer, gastric (stomach) cancer, gastrointestinal carcinoid tumor, gastrointestinal stromal tumors (GIST), ovarian cancer, testicular cancer, extracranial cancers, extragonadal cancers, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kidney cancer, langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer, lymphoma, cutaneous T-Cell cancer, Hodgkin, non-Hodgkin, primary central nervous system (CNS)), macroglobulinemia, Waldenström, male breast cancer, melanoma, merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma, skin cancer, small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

### Embodiment 36

The method of embodiment 34, wherein said pathology comprises a cancer selected from the group consisting of brain cancer, breast cancer, central nervous system cancer, cervical cancer, colorectal cancer, testicular cancer, ovarian cancer, leukemia, a lymphoma, a melanoma, a soft tissue sarcoma, testicular cancer, and thyroid cancer.

### Embodiment 37

The method of embodiment 28, wherein said condition comprises the prevention of transplant rejection.

### Embodiment 38

The method of embodiment 37, wherein said compound or pharmaceutical formulation is used in combination with a calcineurin inhibitor and/or glucocorticoid for the prevention of transplant rejection.

### Embodiment 39

The method of embodiment 37, wherein said compound or pharmaceutical formulation is used in combination with cyclosporine for the prevention of transplant rejection.

### Embodiment 40

The method of embodiment 28, wherein said pathology comprises an autoimmune disease.

### Embodiment 41

The method of embodiment 40, wherein said pathology comprises lupus.

### Embodiment 42

The method of embodiment 40, wherein said pathology comprises multiple sclerosis.

### Embodiment 43

The method of embodiment 28, wherein said pathology comprises a pathology selected from the group consisting of an infection, autism, and a lysosomal storage disease.

### Embodiment 44

The method according to any one of embodiments 28-43, wherein said mammal is a human.

### Embodiment 45

The method according to any one of embodiments 28-43, wherein said mammal is a non-human mammal.

### Embodiment 46

A method of preparing a compound according to any one of embodiments 1, 2, or 3, said method comprising providing the feed starter (1R,4R)-4-hydroxycyclohexanecarboxylic acid in pure chiral form of formula (VII)

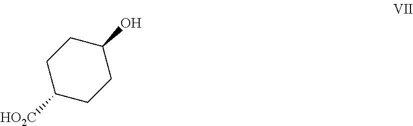

to a rapamycin producing strain of Streptomyces rapamycinicus that has been genetically altered to delete the genes rapI, rapJ, rapK, rapL, rapM, rapN, rapO, and rapQ and conjugated with a plasmid containing rapJ, rapM, rapN, rapO and rapLhis.

### Embodiment 47

A method of preparing a compound according to any one of embodiments 1, 5, or 6, said method comprising providing the feed starter (1R,4R)-4-methoxycyclohexanecarboxylic acid in pure chiral form of formula (VIII)

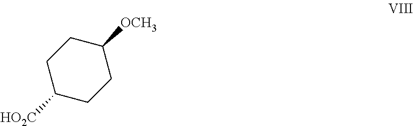 [SMILES: CO[C@H]1CC[C@H](C(=O)O)CC1]

to a rapamycin producing strain of Streptomyces rapamycinicus that has been genetically altered to delete the genes rapI, rapJ, rapK, rapL, rapM, rapN, rapO, and rapQ and conjugated with a plasmid containing rapJ, rapM, rapN, rapO and rapLhis.

### Embodiment 48

A method of preparing a compound according to any one of embodiments 1 or 4, said method comprising providing the feed starter (1R,3R,4R)-3-fluoro-4-hydroxycyclohexane carcarboxylic acid in pure chiral form of formula (IX)

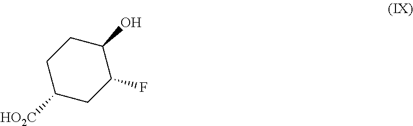 [SMILES: O=C(O)[C@@H]1CC[C@@H](O)[C@H](F)C1]

to a rapamycin producing strain of Streptomyces rapamycinicus that has been genetically altered to delete the genes rapI, rapJ, rapK, rapL, rapM, rapN, rapO, and rapQ and conjugated with a plasmid containing rapJ, rapM, rapN, rapO and rapLhis.

### Embodiment 49

The method according to any one of embodiments 46-48, wherein said strain is S. rapamycinicus strain MG2-10.

### Embodiment 50

A compound according to the formula:

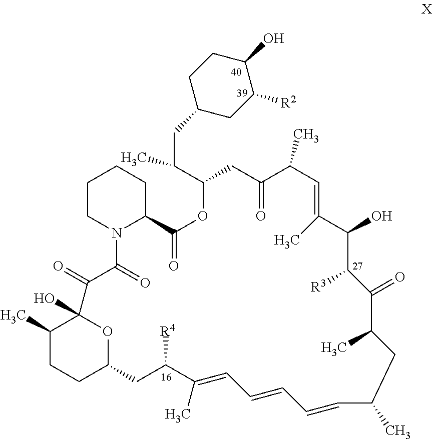

or a pharmaceutically acceptable salt thereof, wherein: R² is H or F; R³ is OH, or OCH₃; and R⁴ is OCH3 or OH.

### Embodiment 51

The compound of embodiment 50, wherein R⁴ is OCH₃.

### Embodiment 52

The compound of embodiment 51, wherein R² is F and R³ is OCH₃.

### Embodiment 53

The compound of embodiment 51, wherein R² is H, and R³ is OH.

### Embodiment 54

The compound of embodiment 50, wherein R² is H, R³ is H, and R⁴ is OH.

### Embodiment 55

A pharmaceutical formulation comprising:
a compound according to any one of embodiments 50-54; and
a pharmaceutically acceptable carrier or excipient.

### Embodiment 56

The formulation of embodiment 55, wherein said formulation is a unit dosage formulation.

### Embodiment 57

The formulation according to any one of embodiments 55-56, wherein said formulation is sterile.

### Embodiment 58

The formulation according to any one of embodiments 55-57, wherein said formulation is formulated for administration via a route selected from the group consisting of administration via inhalation, aerosol administration, intravenous administration, intraarterial administration, oral administration, parenteral delivery, rectal administration, subdural administration, systemic administration, topical administration, transdermal delivery, and vaginal administration.

### Embodiment 59

A compound according to any one of embodiments 50-54, or a pharmaceutical formulation according to any one of embodiments 55-58 for use in one or more of the following: the treatment of a tauopathy, the treatment of an mTORopathy (e.g., such as tuberous sclerosis complex (TSC), focal cortical dysplasia (FCD), ganglioglioma, hemimegalencephaly, neurofibromatosis 1, Sturge-Weber syndrome, Cowden syndrome, PMSE (Polyhydramnios, Megalencephaly, Symptomatic Epilepsy)), the treatment of an mTORopathy associated with epileptic seizures, the treatment of familial multiple discoid fibromas (FMDF), the treatment of an epilepsy/epileptic seizures (both genetic and acquired forms of the disease such as familial focal epilepsies, epileptic spasms, infantile spasms (IS), status epilepticus (SE), temporal lobe epilepsy (PLE) and absence epilepsy), the treatment of rare diseases associated with a dysfunction of mTORC1 activity (e.g., such as lymphangioleiomyomatosis (LAM), Leigh's syndrome, Friedrich's ataxia, Diamond-Blackfan anemia, etc.), the treatment of the treatment of metabolic diseases (e.g., such as obesity, Type II diabetes, etc.), the treatment of autoimmune and inflammatory diseases (e.g., such as Systemic Lupus Erythematosus (SLE), multiple sclerosis (MS) psoriasis, etc.), the treatment of cancer, the treatment of a fungal infection, the treatment of a proliferative disease, the maintenance of immunosuppression, the treatment of transplant rejection, the treatment of traumatic brain injury, the treatment of autism, the treatment of lysosomal storage diseases and the treatment of neurodegenerative diseases associated with an mTORC1 hyperactivity (e.g., such as Parkinson's, Huntington's disease, etc.), and generally treatment of disorders that result in hyperactivation of the mTORC1 pathway.

### Embodiment 60

The compound or pharmaceutical formulation of embodiment 59, for use in the treatment of a tauopathy.

### Embodiment 61

The compound or pharmaceutical formulation of embodiment 60, for use in the treatment of a tauopathy selected from the group consisting of progressive supranuclear palsy, dementia pugilistica (chronic traumatic encephalopathy), frontotemporal dementia, lytico-bodig disease (parkinson-dementia complex of guam), tangle-predominant dementia (with nfts similar to ad, but without plaques), ganglioglioma and gangliocytoma, meningioangiomatosis, subacute sclerosing panencephalitis, lead encephalopathy, tuberous sclerosis, Pick's disease, corticobasal degeneration (tau proteins are deposited in the form of inclusion bodies within swollen or “ballooned” neurons), Alzheimer's disease, Parkinson's disease, Huntington's disease, frontotemporal dementia, frontotemporal lobar degeneration.

### Embodiment 62

The compound or pharmaceutical formulation of embodiment 59, for use in the treatment of an mTORpathy.

### Embodiment 63

The compound or pharmaceutical formulation of embodiment 62, wherein said mTORpathy comprises a pathology selected from the group consisting tuberous sclerosis complex (TSC), focal cortical dysplasia (FCD), ganglioglioma, hemimegalencephaly, neurofibromatosis 1, Sturge-Weber syndrome, Cowden syndrome, and PMSE (Polyhydramnios, Megalencephaly, Symptomatic Epilepsy)).

### Embodiment 64

The compound or pharmaceutical formulation of embodiment 59, for use in the treatment of a pathology selected from the group consisting of epilepsy, neurodegeneration, rare and genetic disease with mTORC1 hyperactivity, metabolic disease, and traumatic brain injury.

### Embodiment 65

The compound or pharmaceutical formulation of embodiment 59, for use in the treatment of cancer.

### Embodiment 66

The compound or pharmaceutical formulation of embodiment 65, wherein said cancer is a cancer selected from the group consisting of acute lymphoblastic leukemia (ALL), acute myeloid leukemia (AML), Adrenocortical carcinoma, kaposi sarcoma, anal cancer, appendix cancer, astrocytomas, atypical teratoid/rhabdoid tumor, bile duct cancer, extrahepatic cancer, bladder cancer, bone cancer, brain stem glioma, astrocytomas, spinal cord tumors, central nervous system atypical teratoid/rhabdoid tumor, central nervous system embryonal tumors, central nervous system germ cell tumors, craniopharyngioma, ependymoma, breast cancer, bronchial tumors, burkitt lymphoma, carcinoid tumors, cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, bile duct cancer, extrahepatic cancer, ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, intraocular melanoma, retinoblastoma, fibrous histiocytoma of bone, malignant, and osteosarcoma, gallbladder cancer, gastric (stomach) cancer, gastrointestinal carcinoid tumor, gastrointestinal stromal tumors (GIST), ovarian cancer, testicular cancer, extracranial cancers, extragonadal cancers, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kidney cancer, langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer, lymphoma, cutaneous T-Cell cancer, Hodgkin, non-Hodgkin, primary central nervous system (CNS)), macroglobulinemia, Waldenström, male breast cancer, melanoma, merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma, skin cancer, small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

### Embodiment 67

The compound or pharmaceutical formulation of embodiment 65, wherein said cancer is a cancer selected from the group consisting of brain cancer, breast cancer, central nervous system cancer, cervical cancer, colorectal cancer, testicular cancer, ovarian cancer, leukemia, a lymphoma, a melanoma, a soft tissue sarcoma, testicular cancer, and thyroid cancer.

### Embodiment 68

The compound or pharmaceutical formulation of embodiment 59, for use in the prevention of transplant rejection.

### Embodiment 69

The compound or pharmaceutical formulation of embodiment 68, for use in combination with a calcineurin inhibitor and/or glucocorticoid for the prevention of transplant rejection.

### Embodiment 70

The compound or pharmaceutical formulation of embodiment 68, for use in combination with cyclosporine for the prevention of transplant rejection.

### Embodiment 71

The compound or pharmaceutical formulation of embodiment 59, for use in the treatment of an autoimmune disease.

### Embodiment 72

The compound or pharmaceutical formulation of embodiment 71, wherein said autoimmune disease comprises lupus.

### Embodiment 73

The compound or pharmaceutical formulation of embodiment 71, wherein said autoimmune disease comprises multiple sclerosis.

### Embodiment 74

The compound or pharmaceutical formulation of embodiment 59, for use in the treatment of an infection, autism, or a lysosomal storage disease.

### Embodiment 75

The compound or pharmaceutical formulation according to any one of embodiments 59-74 for use in the treatment of a human.

### Embodiment 76

The compound or pharmaceutical formulation according to any one of embodiments 59-74 for use in the treatment of a non-human mammal.

### Embodiment 77

A method of treating a mammal for a pathology/condition selected from the group consisting a tauopathy, an mTORopathy (e.g., such as tuberous sclerosis complex (TSC), focal cortical dysplasia (FCD), ganglioglioma, hemimegalencephaly, neurofibromatosis 1, Sturge-Weber syndrome, Cowden syndrome, PMSE (Polyhydramnios, Megalencephaly, Symptomatic Epilepsy)), an mTORopathy associated with epileptic seizures, familial multiple discoid fibromas (FMDF), epilepsy/epileptic seizures (both genetic and acquired forms of the disease such as familial focal epilepsies, epileptic spasms, infantile spasms (IS), status epilepticus (SE), temporal lobe epilepsy (PLE) and absence epilepsy), rare diseases associated with a dysfunction of mTORC1 activity (e.g., such as lymphangioleiomyomatosis (LAM), Leigh's syndrome, Friedrich's ataxia, Diamond-Blackfan anemia, etc.), metabolic diseases (e.g., such as obesity, Type II diabetes, etc.), autoimmune and inflammatory diseases (e.g., such as Systemic Lupus Erythematosus (SLE), multiple sclerosis (MS) psoriasis, etc.), cancer, a fungal infection, a proliferative disease, the maintenance of immunosuppression, the treatment of transplant rejection, a traumatic brain injury, autism, a lysosomal storage disease, a neurodegenerative diseases associated with mTORC1 hyperactivity (e.g., such as Parkinson's, Huntington's disease, etc.), and disorders that result in hyperactivation of the mTORC1 pathway, in a mammal, said method comprising administering said mammal an effective amount of a compound according to any one of embodiments 50-54, or a pharmaceutical formulation according to any one of embodiments 55-58.

### Embodiment 78

The method of embodiment 77, wherein said pathology comprises a tauopathy.

### Embodiment 79

The method of embodiment 78, wherein said pathology comprises a tauopathy selected from the group consisting of progressive supranuclear palsy, dementia pugilistica (chronic traumatic encephalopathy), frontotemporal dementia, lytico-bodig disease (parkinson-dementia complex of guam), tangle-predominant dementia (with nfts similar to ad, but without plaques), ganglioglioma and gangliocytoma, meningioangiomatosis, subacute sclerosing panencephalitis, lead encephalopathy, tuberous sclerosis, Pick's disease, corticobasal degeneration (tau proteins are deposited in the form of inclusion bodies within swollen or “ballooned” neurons), Alzheimer's disease, Parkinson's disease, Huntington's disease, frontotemporal dementia, frontotemporal lobar degeneration.

### Embodiment 80

The method of embodiment 77, wherein said pathology comprises an mTORpathy.

### Embodiment 81

The method of embodiment 80, wherein said mTORpathy comprises a pathology selected from the group consisting tuberous sclerosis complex (TSC), focal cortical dysplasia (FCD), ganglioglioma, hemimegalencephaly, neurofibromatosis 1, Sturge-Weber syndrome, Cowden syndrome, and PMSE (Polyhydramnios, Megalencephaly, Symptomatic Epilepsy)).

### Embodiment 82

The method of embodiment 77, wherein said pathology comprises a pathology selected from the group consisting of epilepsy, neurodegeneration, rare and genetic disease with mTORC1 hyperactivity, metabolic disease, and traumatic brain injury.

### Embodiment 83

The method of embodiment 77, wherein said pathology comprises a cancer.

### Embodiment 84

The method of embodiment 83, wherein said pathology comprises a cancer selected from the group consisting of acute lymphoblastic leukemia (ALL), acute myeloid leukemia (AML), Adrenocortical carcinoma, kaposi sarcoma, anal cancer, appendix cancer, astrocytomas, atypical teratoid/rhabdoid tumor, bile duct cancer, extrahepatic cancer, bladder cancer, bone cancer, brain stem glioma, astrocytomas, spinal cord tumors, central nervous system atypical teratoid/rhabdoid tumor, central nervous system embryonal tumors, central nervous system germ cell tumors, craniopharyngioma, ependymoma, breast cancer, bronchial tumors, burkitt lymphoma, carcinoid tumors, cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, bile duct cancer, extrahepatic cancer, ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, intraocular melanoma, retinoblastoma, fibrous histiocytoma of bone, malignant, and osteosarcoma, gallbladder cancer, gastric (stomach) cancer, gastrointestinal carcinoid tumor, gastrointestinal stromal tumors (GIST), ovarian cancer, testicular cancer, extracranial cancers, extragonadal cancers, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kidney cancer, langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer, lymphoma, cutaneous T-Cell cancer, Hodgkin, non-Hodgkin, primary central nervous system (CNS)), macroglobulinemia, Waldenström, male breast cancer, melanoma, merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma, skin cancer, small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

### Embodiment 85

The method of embodiment 83, wherein said pathology comprises a cancer selected from the group consisting of brain cancer, breast cancer, central nervous system cancer, cervical cancer, colorectal cancer, testicular cancer, ovarian cancer, leukemia, a lymphoma, a melanoma, a soft tissue sarcoma, testicular cancer, and thyroid cancer.

### Embodiment 86

The method of embodiment 77, wherein said condition comprises the prevention of transplant rejection.

### Embodiment 87

The method of embodiment 86, wherein said compound or pharmaceutical formulation is used in combination with a calcineurin inhibitor and/or glucocorticoid for the prevention of transplant rejection.

### Embodiment 88

The method of embodiment 86, wherein said compound or pharmaceutical formulation is used in combination with cyclosporine for the prevention of transplant rejection.

### Embodiment 89

The method of embodiment 77, wherein said pathology comprises an autoimmune disease.

### Embodiment 90

The method of embodiment 89, wherein said pathology comprises lupus.

### Embodiment 91

The method of embodiment 89, wherein said pathology comprises multiple sclerosis.

### Embodiment 92

The method of embodiment 77, wherein said pathology comprises a pathology selected from the group consisting of an infection, autism, and a lysosomal storage disease.

### Embodiment 93

The method according to any one of embodiments 77-92, wherein said mammal is a human.

### Embodiment 94

The method according to any one of embodiments 77-92, wherein said mammal is a non-human mammal.

### Definitions

The terms “subject,” “individual,” and “patient” may be used interchangeably and refer to humans, the as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a rapamycin analog described herein.

As used herein, the term “lupus” includes, without limitation systemic lupus erythrematosis (SLE), lupus nephritis, acute cutaneous lupus erythematosus, subacute cutaneous lupus erythematosus, chronic cutaneous lupus erythematosus, drug-induced lupus erythematosus, neonatal lupus erythematosus,

As used herein, the terms “multiple sclerosis” or “MS” include, without limitation, relapsing remitting, secondary progressive and primary progressive multiple sclerosis.

The terms “tapathy or taupathies” refers to a class of neurodegenerative diseases associated with the pathological aggregation of tau protein, typically in neurofibrillary or gliofibrillary tangles in the human brain (see, e.g., Rizzo et al. (2008) Brain. 131 (Pt 10): 2690-2770). Tangles are believed to be formed by hyperphosphorylation of a microtubule-associated protein known as tau, causing it to aggregate in an insoluble form. Primary tauopathies, e.g., conditions in which neurofibrillary tangles are predominantly observed, include, but are not limited to primary age-related tauopathy (PART)/Neurofibrillary tangle-predominant senile dementia, with NFTs similar to AD, but without plaques (see, e.g., Dickson (2009) Int. J Clin. Exp. Pathol., 3(1): 1-23; Santa-Maria et al. (2012) Acta Neuropathologica. 124(5): 693-704; Jellinger and Attems (2006) Acta Neuropathologica. 113(2): 107-117; and the like), dementia pugilistica (chronic traumatic encephalopathy) (see, e.g., Roberts (1988). Lancet. 2(8626-8627): 1456-1458), progressive supranuclear palsy (see, e.g., Williams et al. (2009). The Lancet Neurology, 8(3): 270-279), corticobasal degeneration, chronic traumatic encephalopathy (see, e.g., Mckee and Cairns (2016) Acta Neuropatholo. 131: 75-86), frontotemporal dementia and parkinsonism linked to chromosome 17 (see, e.g., Selkoe et al. (2002) Ann. rev. Genomics and Human Genetics, 3: 67-99), Lytico-Bodig disease (Parkinson-dementia complex of Guam) (see, e.g., Hof et al. (1994) Acta Neuropathologica. 88(5): 397-404), ganglioglioma and gangliocytoma (see, e.g., Brat et al. (2001) Neuropathol. Appl. Neurobiol., 27(3): 197-205), meningioangiomatosis (see, e.g., Halper et al. (1986) J. Neuropathol. Exp. Neurol., 45(4): 426-446), postencephalitic parkinsonism, subacute sclerosing panencephalitis (see, e.g., Paula-Barbosa et al. (1979) Acta Neuropathologica. 48(2): 157-160), lead encephalopathy, tuberous sclerosis, Hallervorden-Spatz disease, lipofuscinosis (see, e.g., Wisniewski et al. (1979) Annal. Neurol., 5(3): 288-294), and the like.

As used herein, the term “in substantially pure form” means that the compound is provided in a form which is substantially free of other compounds (particularly polyketides or other rapamycin analogues) when produced in fermentation processes, especially a fermentation process involving feeding starter acid as described herein to a rapamycin producing strain that has been genetically altered to remove or inactivate the rapK gene or homologue thereof. For example the purity of the compound is at least 90%, or at least 95%, or at least 98%, or at least 99% as regards the polyketide content of the form in which is it presented. Hence both prior and post formulation as a pharmaceutical product, in various embodiments, the compounds described herein suitably represent at least 90%, or at least 95%, or at least 98%, or least 99% of the polyketide content of the composition or product.

Generally, reference to a certain element such as hydrogen or H is meant to include all isotopes of that element. For example, if an R group is defined to include hydrogen or H, it also includes deuterium and tritium. Accordingly, isotopically labeled compounds are within the scope of this invention.

A pharmaceutically acceptable salt is any salt of the parent compound that is suitable for administration to an animal or human. A pharmaceutically acceptable salt also refers to any salt which may form in vivo as a result of administration of an acid, another salt, or a prodrug which is converted into an acid or salt. A salt comprises one or more ionic forms of the compound, such as a conjugate acid or base, associated with one or more corresponding counterions. Salts can form from or incorporate one or more deprotonated acidic groups (e.g. carboxylic acids), one or more protonated basic groups (e.g. amines), or both (e.g. zwitterions).

The term “substantially pure” or “substantially pure chiral form” when used with respect to enantiomers indicates that one particular enantiomer (e.g. an S enantiomer or an R enantiomer) is substantially free of its stereoisomer(s). In various embodiments substantially pure indicates that a particular enantiomer is at least 70%, or at least 80%, or at least 90%, or at least 95%, or at least 98%, or at least 99% of the purified compound. Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller (1975) J. Chromatogr., 113(3): 283-302). Racemic mixtures of chiral compounds can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

The terms “compound XXX”, “Delos XXX”, “DLXXX”, and “nIDXXX” where XXX is a number (e.g., 390, 384, 405, etc.) are used interchangeably to designate rapalogs described herein. Thus, for example, “compound 390”, “Delos 390”, “DL390”, and “nID390” refer to a rapalog comprising the structure of compound 390, e.g., as shown in FIGS. 2A-2C.

### BRIEF DESCRIPTION OF THE DRAWINGS

FIG. 1 illustrates the structure of rapamycin.

FIGS. 2A-2C illustrate the structure of rapamycin analogues (rapalogs). FIG. 2A: Compound 390 (a.k.a. Delos 390 or nID390). FIG. 2B: Compound 384 (a.k.a. Delos 384 or nID384). FIG. 2C: Compound 405 (a.k.a. Delos 405 or nID405).

FIG. 3. Compounds that fully inhibit mTORC1 and partially inhibit mTORC2 in PC3 cells at 100 nM. Compared to rapamycin, which shows complete inhibition of both mTORC1 (pS6k/S6k) and mTORC2 (pAkt/Akt), the compounds shown herein displayed full mTORC1 inhibition similar to rapamycin, yet only partial inhibition of mTORC2.

FIG. 4. Western blot of rapalogs described herein compared to commercial rapalogs. Protein was collected from PC3 cells following treatment for 24 h at 100 nM with one of five rapalogs, or with EVEROLIMUS®, TEMSIROLIMUS® or rapamycin. Antibodies to pS6 and total S6, Akt and total Akt were used to determine mTORC1 and mTORC2 activity.

FIG. 5. Rapalog compound's mTORC1/2 complex inhibition compared to commercial rapalogs.

FIG. 6. Western blot analysis of normalized mTORC1 (P-S6/total S6) and mTORC2 (P-AKT/Total AKT) inhibition in lung, visceral fat, gastrocnemius muscle, pancreas, heart, soleus, lung, kidney and liver of fasted mice (N=5) following intraperitoneal administration of 8 mg/kg rapamycin, 12 mg/kg compound 390 or vehicle control every other day for 3 weeks.

FIG. 7, panels A-C. Plasma levels of free fatty acids (panel A), cholesterol (panel B) and triglycerides (panel C) following intraperitoneal administration of 8 mg/kg rapamycin, 12 mg/kg compound 390 or vehicle control every other day for 3 weeks (N=5).

FIG. 8, panels A-B. Steady state glucose plasma levels (panel B) and calculated as percent of baseline levels (panel A) determined one week following intraperitoneal administration of 8 mg/kg rapamycin, 12 mg/kg compound 390 or vehicle control every other day for 3 weeks (N=5).

FIG. 9. Glucose challenge response following administration of 2 mg/kg glucose at t=1 week following the initiation of the intraperitoneal administrations of 8 mg/kg rapamycin, 12 mg/kg compound 390 or vehicle control every other day in female mice (N=5).

FIG. 10. Response to insulin challenge following administration of 0.75 IU/kg insulin at t=2 weeks following the initiation of the intraperitoneal administrations of 8 mg/kg rapamycin, 12 mg/kg compound 390 or vehicle control every other day in female mice (N=5).

FIG. 11. Glucose challenge response following administration of 1 mg/kg glucose at t=2 weeks following the initiation of the intraperitoneal administrations of 8 mg/kg rapamycin (N=18), 12 mg/kg compound 390 (N=9), or vehicle control (N=18) every other day in male C57BL/6J mice. p values as specified in the figure, two-tailed t-test.

FIG. 12. Pyruvate challenge following administration of 2 mg/kg pyruvate at t=3 weeks following the initiation of the intraperitoneal administrations of 8 mg/kg rapamycin (N=18), 12 mg/kg compound 390 (N=9), or vehicle control (N=18) every other day in male C57BL/6J mice. p values as specified in the figure, two-tailed t-test.

FIG. 13. Fat mass following four weeks of intraperitoneal administrations of 8 mg/kg rapamycin (N=18), 12 mg/kg compound 390 (N=9) or vehicle control (N=18) every other day in male C57BL/6J mice. Body composition was measured in before and after treatment using an EchoMRI 3-in-1 body composition analyzer. P values as specified in the figure.

FIG. 14A-14C. Molecular impact of rapamycin and compound 390 in the liver (FIG. 14A), muscle (FIG. 14B), and brain (FIG. 14C) following chronic (four weeks) of intraperitoneal administrations of 8 mg/kg rapamycin (N=18), 12 mg/kg compound 390 (N=9) or vehicle control (N=18) every other day in male C57BL/6J mice.

FIG. 15. Molecular impact of rapamycin and compound 390 in the integrity of mTORC2 assembly in the liver following chronic (four weeks) of intraperitoneal administrations of 8 mg/kg rapamycin (N=18), 12 mg/kg compound 390 (N=9), or vehicle control (N=18) every other day in male C57BL/6J mice.

FIG. 16A-16B. Levels of total and pTau (pThr¹⁸¹) in the soluble fraction in the cortex (FIG. 16A) and hippocampus (FIG. 16B) of Tg4510 animals receiving either vehicle control (N=15) or 12 mg/kg compound 390 (DL390) following intraperitoneal administration (N=15) for four weeks.

FIG. 17A-17B. Levels of pTau in the insoluble fraction determined by three different antibodies (pSer²⁰²/Thr²⁰⁵, pThr²³⁵, pThr¹⁸¹) in the cortex (FIG. 17A) and hippocampus (FIG. 17B) of Tg4510 animals receiving either vehicle control (N=15) or 12 mg/kg compound 390 (DL390) following intraperitoneal administration (N=15) for four weeks. Data analysis: Unpaired t-test. ****=p<0.0001; ***=p<0.001; **=p<0.01.

FIG. 18. Levels of autophagy markers Beclin-1, Vps34, pULK1/ULK, LC3B-II, LC3B-I and p62 in the cortehippocampus of Tg4510 animals receiving either vehicle control (N=15) or 12 mg/kg compound 390 (DL390) following intraperitoneal administration (N=15) for four weeks. Data analysis: One-way ANOVA followed by Tukey's comparison test. ****=p<0.0001, ***=p<0.001, **=p<0.01, *=p<0.05 (compared to wt, vehicle). ####=p<0.0001, ###=p<0.001, ##=p<0.01, #=p<0.05 (compared to Tg4510, vehicle).

FIG. 19. Average distance travelled on acquisition days. Data are expressed as the mean±SEM. *p<0.05 compared to WT-vehicle; #p<0.05 compared to Tg4510-vehicle Tg4510.

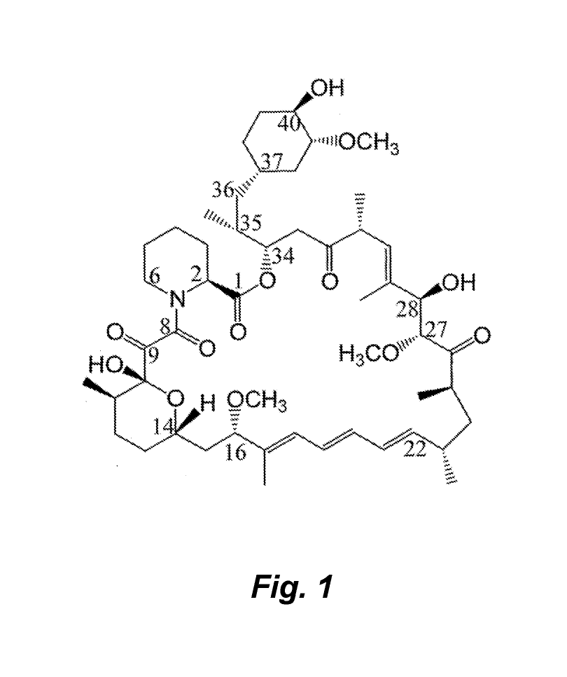

### DETAILED DESCRIPTION

In various embodiments rapamycin analogs are provided that are believed to provide an improved therapeutic window, e.g., as compared to rapamycin. In particular, it is believed the compounds identified herein have reduced inhibition at mTORC2 as compared to an equivalent dose of rapamycin, while still affording inhibitory activity at mTORC1. In certain embodiments the compounds show inhibitory activity comparable to or greater than rapamycin a mTORC1 at the same dosage while showing lower (or no) inhibitory activity at mTORC2.

The compounds described herein were obtained by synthesizing a library of unique rapamycin analogs (rapalogs) and screening that library in PC3 cells to identify rapalogs, that exhibited various degrees of mTORC1 selective inhibitory action (compared to rapamycin). A subset of these rapalogs was selected and the dose-responsiveness of their mTORC1 and mTORC2 inhibitory action was examined, in order to identify compounds that are as effective at inhibitor mTORC1 as rapamycin without inhibiting mTORC2. This approach resulted, inter alia, the compound described herein.

It is believed the rapamycin analogs described herein find use in the treatment of lupus, the treatment of multiple sclerosis, the treatment of a fungal infection, the treatment of chronic plaque psoriasis, the treatment of a proliferative disease (including, but not limited to cancers), the maintenance of immunosuppression (e.g., after organ transplant), the treatment of epileptic seizures, the treatment of tuberous sclerosis complex (TSC), the treatment of multiple sclerosis, the treatment of familial multiple discoid fibromas (FMDF), the treatment of cardiovascular disease, the treatment of various autoimmune diseases, the treatment of various neurodegenerative diseases including, but not limited to tauopathies (conditions in which neurofibrillary tangles are commonly observed). Illustrative tauopathies include, but are not limited to progressive supranuclear palsy, dementia pugilistica (chronic traumatic encephalopathy), frontotemporal dementia, lytico-bodig disease (parkinson-dementia complex of guam), tangle-predominant dementia (with nfts similar to ad, but without plaques), ganglioglioma and gangliocytoma, meningioangiomatosis, subacute sclerosing panencephalitis, lead encephalopathy, tuberous sclerosis, Pick's disease, corticobasal degeneration (tau proteins are deposited in the form of inclusion bodies within swollen or “ballooned” neurons), Alzheimer disease, Huntington's disease, frontotemporal dementia, frontotemporal lobar degeneration, and the like.

### Rapamycin Analogs.

In various embodiments the rapamycin analogues include a compound of formula (I):

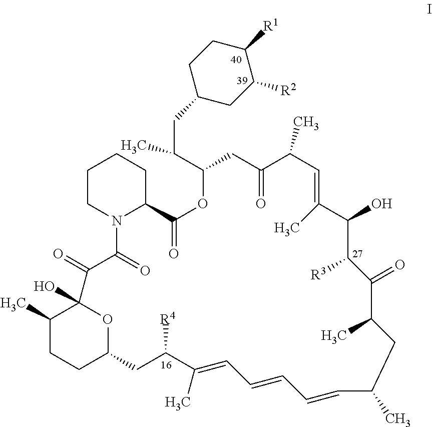

or a pharmaceutically acceptable salt thereof, where R¹ is OH or OCH₃, R² is H or F, R³ is H, OH, or OCH₃; and R⁴ is OH or OCH₃.

In certain embodiments the compound is in pure chiral form as a single diastereomer of formula II:

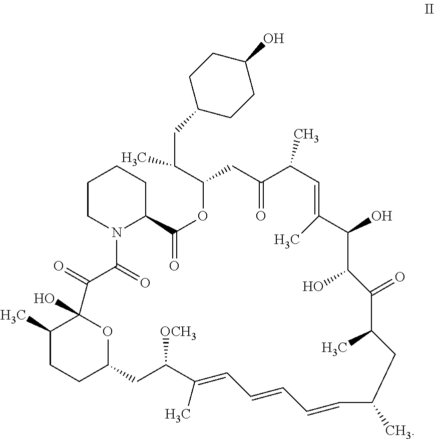 [SMILES: CO[C@H]1C[C@@H]2CC[C@@H](C)[C@@](O)(O2)C(=O)C(=O)N2CCCC[C@H]2C(=O)O[C@H]([C@H](C)C[C@H]2CC[C@H](O)CC2)CC(=O)[C@H](C)/C=C(\C)[C@@H](O)[C@@H](O)C(=O)[C@H](C)C[C@H](C)/C=C/C=C/C=C/1C]

In certain embodiments the compound is in pure chiral form as a single diastereomer of formula III:

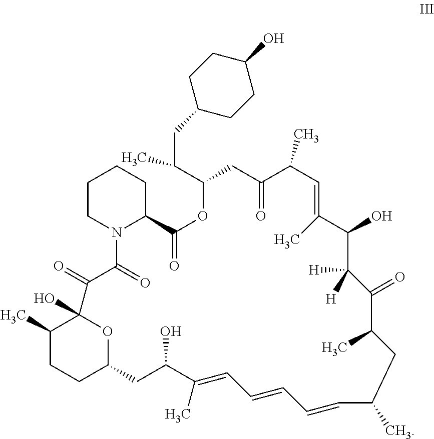

In certain embodiments the compound is in substantially pure chiral form as a single diastereomer of formula IV:

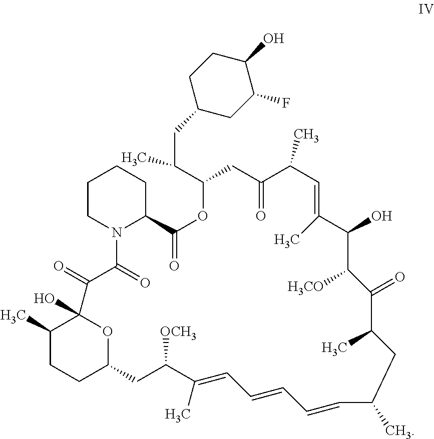 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](F)C2)CC(=O)[C@H](C)/C=C(\C)[C@@H](O)[C@@H](OC)C(=O)[C@H](C)C[C@H](C)/C=C/C=C/C=C/1C]

In certain embodiments the compound is in substantially pure chiral form as a single diastereomer of formula V:

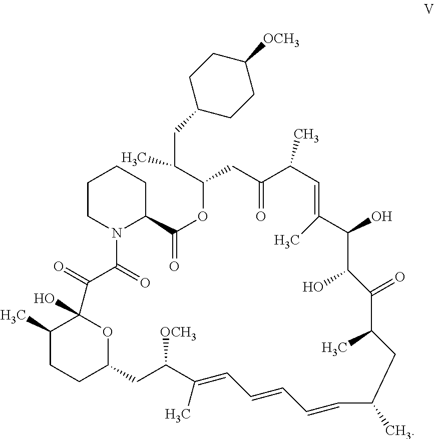 [SMILES: CO[C@H]1C[C@@H]2CC[C@@H](C)[C@@](O)(O2)C(=O)C(=O)N2CCCC[C@H]2C(=O)O[C@H]([C@H](C)C[C@H]2CC[C@H](OC)CC2)CC(=O)[C@H](C)/C=C(\C)[C@@H](O)[C@@H](O)C(=O)[C@H](C)C[C@H](C)/C=C/C=C/C=C/1C]

In certain embodiments the compound is in substantially pure chiral form as a single diastereomer of formula VI:

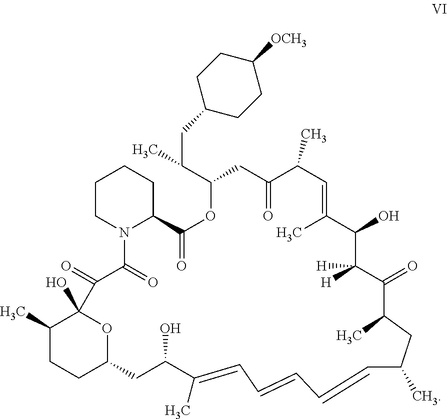

In various embodiments the rapamycin analogues include a compound of formula (X):

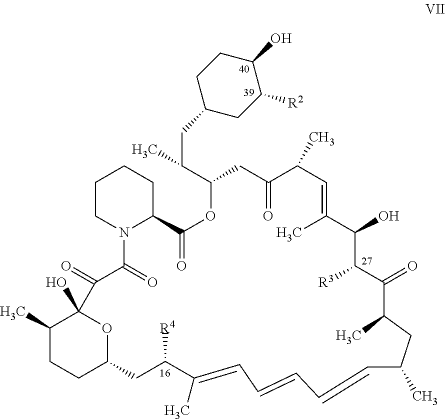

or a pharmaceutically acceptable salt thereof, where R² is H or F, R³ is OH, or OCH₃; and R⁴ is OCH3 or OH. In certain embodiments R⁴ is OCH₃. In certain embodiments R⁴ is OCH₃, R² is F, and R³ is OCH₃. In certain embodiments R⁴ is OCH₃, R² is H, and R³ is OH. In certain embodiments R² is H, R³ is H, and R⁴ is OH. In various embodiments the compounds of Formula VII are present as a racemic mixture.

Without being bound to a particular theory it is believed these compounds are, in various embodiments, preferential mTORC1 inhibitor.

### Production of Rapamycin Analogs.

In various embodiments the rapamycin analogs described herein are produced by the use of a recombinant host strain of Streptomyces (e.g., S. hygroscopicus) containing genomic detections of one or more of genes selected from the group consisting of rapQ, rapO, rapN, rapM, rapL, rapK, rapJ, rapI introduced into S. hygroscopicus and complementation or partial complementation by expressing single genes or combinations of genes, including but not limited to rapK, rapI, rapQ, rapM, the contiguous genes rapN and O (herein designated as rapN/O), rapL and rapJ, in gene cassettes. The method typically further involves culturing the recombinant host strain, and optionally isolating the rapamycin analogues produced. Thus, for example, as illustrated in PCT Publication No: W) 2004/007709 (PCT/GB2003/003230) the recombinant strain MG2-10[pSGsetrapK], produced by complementation of the genomic deletion strain S. hygroscopicus MG2-10, with rapK, was cultured to produce 9-deoxo-16-O-desmethyl-27-desmethoxy-39-O-desmethyl-rapamycin (prerapamycin).

As noted above, the strategy typically involves the integration of a vector comprising a sub-set of genes including, but not limited to, rapK, rapI, rapQ, rapM, rapN, rapO, rapL and rapJ into the S. hygroscopicus deletion mutant above. Such integration may be performed using a variety of available integration functions including but not limited to: ΦC31-based vectors, vectors based on pSAM2 integrase (e.g. in pPM927 (Smovkina et al. (1990) Gene 94: 53-59), R4 integrase (e.g., in pAT98 (Matsuura et al. (1996 J Bad. 178(11): 3374-3376), OVWB integrase (e.g., in pKT02 (Van Mellaert et al. (1998) Microbiology 144:3351-3358, BT1 integrase (e.g., pRT801), and L5 integrase (e.g., Lee et al. (1991) Proc. Natl. Acad. Sci. USA, 88:3111-3115).

In some cases the integration is facilitated by alteration of the host strain, e.g., by addition of the specific attB site for the integrase to enable high efficiency integration. In certain embodiments replicating vectors can also be used, either as replacements to, or in addition to ΦC31-based vectors. These include, but are not limited to, vectors based on pIJ101 (e.g., plJ487, Kieser et al. (2000) Practical Streptomyces Genetics, John Innes Foundation ISBN 0-7084-0623-8), pSG5 (e.g. pKC1139, Bierman et al. (1992) Gene 116: 43-49) and SCP2* (e.g., plJ698, Kieser et al. (2000), supra.).

Although the introduction of gene cassettes into S. hygroscopicus has been exemplified using the ΦBT1 and the ΦC31 site-specific integration functions, those skilled in the art will appreciate that there are a number of different strategies described in the literature, including those mentioned above that could also be used to introduce such gene cassettes into prokaryotic, or more preferably actinomycete, host strains. These include the use of alternative site-specific integration vectors as described above and in the following articles (Kieser et al. (2000), supra.; Van Mellaert et al. (1998) Microbiology 144:3351-3358; Lee et al. (1991) Proc. Natl. Acad. Sci. USA, 88:3111-3115; Smovkina et al. (1990) Gene 94: 53-59; Matsuura et al. (1996 J Bad. 178(11): 3374-3376). Alternatively, plasmids containing the gene cassettes may be integrated into a neutral site on the chromosome using homologous recombination sites. Further, for a number of actinomycete host strains, including S. hygroscopicus, the gene cassettes may be introduced on self-replicating plasmids (Kieser et al. (2000), supra.; WO 1998/001571).

Typically, a gene cassette is used for the complementation of the recombinant S. hygroscopicus deletion strains. Methods of constructing gene cassettes and their heterologous use to produce hybrid glycosylated macrolides have been previously described (Gaisser et al. (2002) Mol. Microbiol. 44: 771-781; PCT Pub. Nos. WO 2001/079520, WO 2003/0048375, and WO 2004/007709). In certain embodiments the gene cassette is assembled directly in an expression vector rather than pre-assembling the genes in pUC18/19 plasmids, thus providing a more rapid cloning procedure.

The approach is exemplified in PCT Pub. No. WO 2004/007709. As described herein, a suitable vector (for example but without limitation pSGLit1) can be constructed for use in the construction of said gene cassettes, where a suitable restriction site (for example but without limitation XbaI), sensitive to dam methylation is inserted 5′ to the gene(s) of interest and a second restriction site (for example XbaI) can be inserted 3′ to the genes of interest. The skilled artisan will appreciate that other restriction sites may be used as an alternative to XbaI and that the methylation sensitive site may be 5′ or 3′ of the gene(s) of interest.

The cloning strategy also allows the introduction of a histidine tag in combination with a terminator sequence 3′ of the gene cassette to enhance gene expression. Those skilled in the art will appreciate other terminator sequences could be used.

In certain embodiments various different promotor sequences can be used in the assembled gene cassette to optimize gene expression. Using these methods (e.g., as further described in WO 2004/007709) S. hygroscopicus deletion strains, the deletion comprising, but not limited to, a gene or a sub-set of the genes rapQ, rapN/O, rapM, rapL, rapK, rapJ and rapI can readily be constructed. In various embodiments the gene cassettes for complementation or partial complementation would generally comprise single genes or a plurality of genes selected from the sub-set of the genes deleted.

In another approach, the rapamycin analogues described herein can be obtained by a process comprising the steps of:

a) constructing a deletion strain, where the deletion(s) include, but not limited to, the genes rapK, rapQ, rapN/O, rapM, rapL, rapJ and rapI, or a sub-set thereof;

b) culturing the strain under conditions suitable for polyketide production;

c) optionally, isolating the rapamycin analogue intermediate produced;

d) constructing a biotransformation strain containing a gene cassette comprising all or a sub-set of the genes deleted;

e) feeding the rapamycin analogue intermediate in culture supernatant or isolated as in step c) to a culture of the biotransformation strain under suitable biotransformation conditions; and

f) optionally isolating the rapamycin analogue produced.

It is well known to those skilled in the art that polyketide gene clusters may be expressed in heterologous hosts (Pfeifer and Khosla, 2001). Accordingly, suitable host strains for the construction of the biotransformation strain include the native host strain in which the rapamycin biosynthetic gene cluster has been deleted, or substantially deleted or inactivated, so as to abolish polyketide synthesis, or a heterologous host strain. Methods for the expressing of gene cassettes comprising one or a plurality of modifying or precursor supply genes in heterologous hosts are described in WO 2001/079520. In this context heterologous hosts suitable for biotransformation of the rapamycin anlaogues include, but are not limited to, S. hygroscopicus, S. hygroscopicus sp., S. hygroscopicus var. ascomyceticus, Streptomyces tsukubaensis, Streptomyces coelicolor, Streptomyces lividans, Saccharopolyspora erythraea, Streptomyces fradiae, Streptomyces avermitilis, Streptomyces cinnamonensis, Streptomyces rimosus, Streptomyces albus, Streptomyces griseofuscus, Streptomyces longisporoflavus, Streptomyces venezuelae, Micromonospora griseorubida, Amycolatopsis mediterranei, Escherichia coli and Actinoplanes sp. N902-109, and the like.

The close structural relationship between rapamycin and FK506, FK520, FK523, ‘hyg’, meridamycin, antascomicin, FK525 and tsukubamycin, among others, and the established homologies between genes involved in the biosynthesis of rapamycin and FK506 and FK520 (vide supra), renders the application of the synthesis methods described herein straightforward in these closely related systems.

It has been demonstrated that rapK is involved in the supply of the biosynthetic precursors (e.g., 4,5-dihydroxycyclohex-1-ene carboxylic acid starter) for rapamycin production. Moreover, deletion or inactivation of rapK or a rapK homologue provides a strain lacking in competition between the natural starter unit and fed non-natural starter units. In another aspect, the invention provides, a method for the efficient incorporation of fed acids including, but not limited to those described below. Thus, for example, Table 1 illustrates various starter units that can be used to produce the rapamycin analogs described herein.

TABLE 1
Illustrative, but non-limiting fed starter units and the
resulting substituent attached to carbon 36.

| Starter acid feed | At Carbon 36 |
|---|---|
| 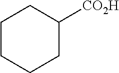 | 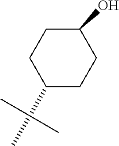 |
| 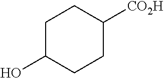 | 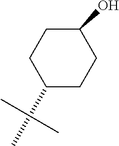 |
| 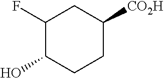 [SMILES: O=C(O)[C@H]1CC[C@H](O)C(F)C1] | 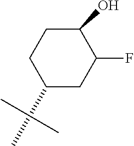 |
| 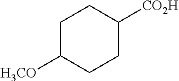 [SMILES: COC1CCC(C(=O)O)CC1] | 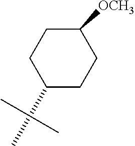 |
| 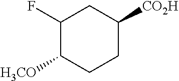 [SMILES: CO[C@H]1CC[C@H](C(=O)O)CC1F] | 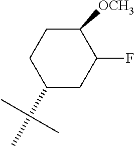 |

While deletion of rapK to facilitate incorporation of these starter units is a typical approach in the production of the compounds described herein, it will be recognized that other methods are available to remove the competition between the endogenously produced natural starter unit and the alternative starter acid analogues fed. For example, it is possible to disrupt the biosynthesis of the natural 4,5-dihydroxycyclohex-1-enecarboxylic acid starter unit. This may be achieved by deletion or inactivation 6f one or more of the genes involved in the biosynthesis of the natural 4,5-dihydroxycyclohex-1-enecarboxylic acid starter unit from shikimic acid (Lowden et al. (2001) Angewandte Chemie—International Edition 40: 777-779) or the biosynthesis of shikimic acid itself. In the latter case, it may be necessary to supplement cultures with aromatic amino acids (phenyl alanine, tyrosine, tryptophan). Alternatively, endogenous production of the natural 4,5-ihydroxycyclohex-1-ene carboxylic acid starter unit may be suppressed by the addition of a chemical inhibitor of shikimic acid biosynthesis.

In various embodiments, the methods described herein produce a racemic mixture of the desired rapamycin analogs and such racemic mixtures can readily be used in the pharmaceutical formulations and treatment methods described herein.

However, in certain embodiments a pure chiral form of the molecule as a single diastereomer is desired. Accordingly, in certain embodiments, methods of preparing a compound in pure chiral form as a single diastereomer of formula II or III are provided where the methods involve providing the feed starter (1R,4R)-4-hydroxycyclohexanecarboxylic acid in pure chiral form of formula (VII)

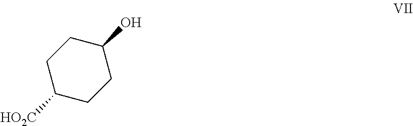

to a rapamycin producing strain of Streptomyces (e.g., Streptomyces rapamycinicus) that has been genetically altered to delete the genes rapI, rapJ, rapK, rapL, rapM, rapN, rapO, and rapQ and conjugated with a plasmid containing rapJ, rapM, rapN, rapO and rapLhis.

In certain embodiments, a method of preparing a compound in pure chiral form as a single diastereomer of formula V is provided where the method comprises providing the feed starter (1R,4R)-4-methoxycyclohexanecarboxylic acid in pure chiral form of formula (VIII)

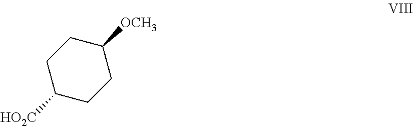 [SMILES: CO[C@H]1CC[C@H](C(=O)O)CC1]

to a rapamycin producing strain of Streptomyces (e.g., Streptomyces rapamycinicus) that has been genetically altered to delete the genes rapI, rapJ, rapK, rapL, rapM, rapN, rapO, and rapQ and conjugated with a plasmid containing rapJ, rapM, rapN, rapO and rapLhis.

In certain embodiments, a method of preparing a compound in pure chiral form as a single diastereomer of formula IV is provided where the method involves providing the feed starter (1R,3R,4R)-3-fluoro-4-hydroxycyclohexane carcarboxylic acid in pure chiral form of formula (IX)

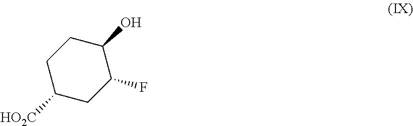 [SMILES: O=C(O)[C@@H]1CC[C@@H](O)[C@H](F)C1]

to a rapamycin producing strain of Streptomyces (e.g., Streptomyces rapamycinicus) that has been genetically altered to delete the genes rapI, rapJ, rapK, rapL, rapM, rapN, rapO, and rapQ and conjugated with a plasmid containing rapJ, rapM, rapN, rapO and rapLhis.

Culture conditions are as described in WO 2004/007709 and in Example 1 herein.

The desired rapamycin analog(s) can be purified using methods known to those of skill in the art, e.g., as described in WO 2004/007709 and herein in Example 1.

It will be recognized that these preparation methods are illustrative and not limiting. Using the teaching provided herein, numerous other methods of producing the rapamycin analogs described herein will be available to one of skill in the art.

### Pharmaceutical Formulations.

In certain embodiments one or more of the rapamycin analogs described herein (e.g., compound 390, compound 405, compound 384, and the like) are administered to a mammal in need thereof, e.g., to a mammal at risk for or suffering from a pathology such as lupus, a fungal infection, chronic plaque psoriasis, a proliferative disease (including, but not limited to cancer), to a mammal in need of the maintenance of immunosuppression (e.g., after organ transplant), for treatment of epileptic seizures, for the treatment of tuberous sclerosis complex (TSC), for the treatment of familial multiple discoid fibromas (FMDF), for the treatment of cardiovascular disease, for the treatment of various autoimmune diseases, for the treatment of various neurodegenerative diseases including, but not limited to tauopathies. In certain embodiments the rapamycin analogs are administered to prevent or delay the onset of the pathology, and/or to ameliorate one or more symptoms of the pathology, and/or to prevent or delay the progression of the pathology, and/or to cure the pathology or induce remission.

The rapamycin analog(s) can be administered in the “native” form or, if desired, in the form of salts, esters, amides, prodrugs, clathrates, derivatives, and the like, provided the salt, ester, amide, prodrug, clathrate, or derivative is pharmacologically suitable, e.g., effective in treatment of a pathology and/or various symptoms thereof, e.g., as described herein. Salts, esters, amides, clathrates, prodrugs and other derivatives of the rapamycin analogs can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience, and as described above.

For example, a pharmaceutically acceptable salt can be prepared for any of the rapamycin analogs described herein having a functionality capable of forming a salt. A pharmaceutically acceptable salt is any salt that retains the activity of the parent compound and does not impart any deleterious or untoward effect on the subject to which it is administered and in the context in which it is administered.

In various embodiments pharmaceutically acceptable salts may be derived from organic or inorganic bases. The salt may be a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules.

Methods of formulating pharmaceutically rapamycin analogs as salts, esters, amide, prodrugs, and the like are well known to those of skill in the art. For example, salts can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain particularly preferred acid addition salts of the rapamycin analogs herein include halide salts, such as may be prepared using hydrochloric or hydrobromic acids. Conversely, preparation of basic salts of the rapamycin analogs of this invention are prepared in a similar manner using a pharmaceutically acceptable base such as sodium hydroxide, potassium hydroxide, ammonium hydroxide, calcium hydroxide, trimethylamine, or the like. Particularly preferred basic salts include alkali metal salts, e.g., the sodium salt, and copper salts.

For the preparation of salt forms of basic drugs, the pKa of the counterion is preferably at least about 2 pH units lower than the pKa of the drug. Similarly, for the preparation of salt forms of acidic drugs, the pKa of the counterion is preferably at least about 2 pH units higher than the pKa of the drug. This permits the counterion to bring the solution's pH to a level lower than the pHₘₐₓ to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (i.e., break down into the individual entities of drug and counterion) in an aqueous environment.

Preferably, the counterion is a pharmaceutically acceptable counterion. Suitable anionic salt forms include, but are not limited to acetate, benzenesulfonate, benzoate, benzylate, bicarbonate, bitartrate, bitartrate, bromide, calcium edetate, camsylateh, carbonate, chloride, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionatei, lactate, lactobionate, malate, maleate, mandelate, mesylate, methylbromide, methylnitrate, methylsulfate, mucate, napsylate, nitrate, pamoate (embonate), pantothenate, phosphate and diphosphate, polygalacturonate, salicylate and disalicylate, stearate, subacetate, succinate, sulfate, tannate, tartrate, teoclate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like. Suitable cationic salt forms include, but are not limited to Benzathine, chloroprocaine, choline, diethanolamine, ethylenediamine, meglumine, procaine, aluminum, calcium, lithium, magnesium, potassium, sodium, zinc, and the like.

Preparation of esters typically involves functionalization of hydroxyl and/or carboxyl groups that are present within the molecular structure of the rapamycin analog. In certain embodiments, the esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

In various embodiments, the rapamycin analogs described herein (e.g., compound 390, compound 405, compound 384, and the like) are useful for parenteral administration, topical administration, oral administration, nasal administration (or otherwise inhaled), rectal administration, or local administration, such as by aerosol or transdermally, for prophylactic and/or therapeutic treatment of one or more of the pathologies/indications described herein (e.g., pathologies characterized by excess amyloid plaque formation and/or deposition or undesired amyloid or pre-amyloid processing).

The rapamycin analogs described herein can also be combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds, particularly of use in the preparation of tablets, capsules, gel caps, and the like include, but are not limited to binders, diluent/fillers, disentegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g. calcium carbonate, carboxymethylcellulose calcium, sodium starch glycollate, crospovidone etc.), a binder (e.g. alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the active component or components (e.g., compound 390, compound 405, compound 384, and the like)) and the resulting composition is compressed. Where necessary the compressed product is coated, e.g., using known methods for masking the taste or for enteric dissolution or sustained release. Suitable coating materials include, but are not limited to ethyl-cellulose, hydroxymethylcellulose, POLYOX® yethylene glycol, cellulose acetate phthalate, hydroxypropylmethylcellulose phthalate, and Eudragit (Rohm & Haas, Germany; methacrylic-acrylic copolymer).

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectibles, implantable sustained-release formulations, mucoadherent films, topical varnishes, lipid complexes, etc.

Pharmaceutical compositions comprising the rapamycin analogs described herein (e.g., compound 390, compound 405, compound 384, and the like) can be manufactured by means of conventional mixing, dissolving, granulating, dragee-making, levigating, emulsifying, encapsulating, entrapping or lyophilizing processes. Pharmaceutical compositions can be formulated in a conventional manner using one or more physiologically acceptable carriers, diluents, excipients or auxiliaries that facilitate processing of the active agent(s) into preparations that can be used pharmaceutically. Proper formulation is dependent upon the route of administration chosen.

In certain embodiments, the active agents described herein are formulated for oral administration. For oral administration, suitable formulations can be readily formulated by combining the active agent(s) with pharmaceutically acceptable carriers suitable for oral delivery well known in the art. Such carriers enable the active agent(s) described herein to be formulated as tablets, pills, dragees, caplets, lizenges, gelcaps, capsules, liquids, gels, syrups, slurries, suspensions and the like, for oral ingestion by a patient to be treated. For oral solid formulations such as, for example, powders, capsules and tablets, suitable excipients can include fillers such as sugars (e.g., lactose, sucrose, mannitol and sorbitol), cellulose preparations (e.g., maize starch, wheat starch, rice starch, potato starch, gelatin, gum tragacanth, methyl cellulose, hydroxypropylmethyl-cellulose, sodium carboxymethylcellulose), synthetic polymers (e.g., polyvinylpyrrolidone (PVP)), granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques. The preparation of enteric-coated particles is disclosed for example in U.S. Pat. Nos. 4,786,505 and 4,853,230.

For administration by inhalation, the active agent(s) are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator may be formulated containing a powder mix of the compound and a suitable powder base such as lactose or starch.

In various embodiments the active agent(s) can be formulated in rectal or vaginal compositions such as suppositories or retention enemas, e.g., containing conventional suppository bases such as cocoa butter or other glycerides. Methods of formulating active agents for rectal or vaginal delivery are well known to those of skill in the art (see, e.g., Allen (2007) Suppositories, Pharmaceutical Press) and typically involve combining the active agents with a suitable base (e.g., hydrophilic (PEG), lipophilic materials such as cocoa butter or Witepsol W45), amphiphilic materials such as Suppocire AP and polyglycolized glyceride, and the like). The base is selected and compounded for a desired melting/delivery profile.

For topical administration the rapamycin analogs described herein (e.g., compound 390, compound 405, compound 384, and the like) can be formulated as solutions, gels, ointments, creams, suspensions, and the like as are well-known in the art.

In certain embodiments the rapamycin analogs described herein are formulated for systemic administration (e.g., as an injectable) in accordance with standard methods well known to those of skill in the art. Systemic formulations include, but are not limited to, those designed for administration by injection, e.g. subcutaneous, intravenous, intramuscular, intrathecal or intraperitoneal injection, as well as those designed for transdermal, transmucosal oral or pulmonary administration. For injection, the active agents described herein can be formulated in aqueous solutions, preferably in physiologically compatible buffers such as Hanks solution, Ringer's solution, or physiological saline buffer and/or in certain emulsion formulations. The solution(s) can contain formulatory agents such as suspending, stabilizing and/or dispersing agents. In certain embodiments the active agent(s) can be provided in powder form for constitution with a suitable vehicle, e.g., sterile pyrogen-free water, before use. For transmucosal administration, and/or for blood/brain barrier passage, penetrants appropriate to the barrier to be permeated can be used in the formulation. Such penetrants are generally known in the art. Injectable formulations and inhalable formulations are generally provided as a sterile or substantially sterile formulation.

In addition to the formulations described previously, the active agent(s) may also be formulated as a depot preparations. Such long acting formulations can be administered by implantation (for example subcutaneously or intramuscularly) or by intramuscular injection. Thus, for example, the active agent(s) may be formulated with suitable polymeric or hydrophobic materials (for example as an emulsion in an acceptable oil) or ion exchange resins, or as sparingly soluble derivatives, for example, as a sparingly soluble salt.

In certain embodiments the active agent(s) described herein can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one illustrative embodiment, the reservoir comprises a polymeric matrix of a pharmaceutically acceptable contact adhesive material that serves to affix the system to the skin during drug delivery. Examples of suitable skin contact adhesive materials include, but are not limited to polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

Alternatively, other pharmaceutical delivery systems can be employed. For example, liposomes, emulsions, and microemulsions/nanoemulsions are well known examples of delivery vehicles that may be used to protect and deliver pharmaceutically active compounds. Certain organic solvents such as dimethylsulfoxide also can be employed, although usually at the cost of greater toxicity.

In certain embodiments the rapamycin analogs described herein (e.g., compound 390, compound 405, compound 384, and the like) are formulated in a nanoemulsion. Nanoemulsions include, but are not limited to oil in water (O/W) nanoemulsions, and water in oil (W/O) nanoemulsions. Nanoemulsions can be defined as emulsions with mean droplet diameters ranging from about 20 to about 1000 nm. Usually, the average droplet size is between about 20 nm or 50 nm and about 500 nm. The terms sub-micron emulsion (SME) and mini-emulsion are used as synonyms.

Illustrative oil in water (O/W) nanoemulsions include, but are not limited to: Surfactant micelles—micelles composed of small molecules surfactants or detergents (e.g., SDS/PBS/2-propanol); Polymer micelles—micelles composed of polymer, copolymer, or block copolymer surfactants (e.g., Pluronic L64/PBS/2-propanol); Blended micelles—micelles in which there is more than one surfactant component or in which one of the liquid phases (generally an alcohol or fatty acid compound) participates in the formation of the micelle (e.g., octanoic acid/PB S/EtOH); Integral micelles—blended micelles in which the active agent(s) serve as an auxiliary surfactant, forming an integral part of the micelle; and Pickering (solid phase) emulsions—emulsions in which the active agent(s) are associated with the exterior of a solid nanoparticle (e.g., polystyrene nanoparticles/PBS/no oil phase).

Illustrative water in oil (W/O) nanoemulsions include, but are not limited to: Surfactant micelles—micelles composed of small molecules surfactants or detergents (e.g., dioctyl sulfosuccinate/PBS/2-propanol, isopropylmyristate/PBS/2-propanol, etc.); Polymer micelles—micelles composed of polymer, copolymer, or block copolymer surfactants (e.g., PLURONIC® L121/PBS/2-propanol); Blended micelles—micelles in which there is more than one surfactant component or in which one of the liquid phases (generally an alcohol or fatty acid compound) participates in the formation of the micelle (e.g., capric/caprylic diglyceride/PBS/EtOH); Integral micelles—blended micelles in which the active agent(s) serve as an auxiliary surfactant, forming an integral part of the micelle (e.g., active agent/PBS/polypropylene glycol); and Pickering (solid phase) emulsions—emulsions in which the active agent(s) are associated with the exterior of a solid nanoparticle (e.g., chitosan nanoparticles/no aqueous phase/mineral oil).

As indicated above, in certain embodiments the nanoemulsions comprise one or more surfactants or detergents. In some embodiments the surfactant is a non-anionic detergent (e.g., a polysorbate surfactant, a polyoxyethylene ether, etc.). Surfactants that find use in the present invention include, but are not limited to surfactants such as the TWEEN®, TRITON®, and TYLOXAPOL® families of compounds.

In certain embodiments the emulsions further comprise one or more cationic halogen containing compounds, including but not limited to, cetylpyridinium chloride. In still further embodiments, the compositions further comprise one or more compounds that increase the interaction (“interaction enhancers”) of the composition with microorganisms (e.g., chelating agents like ethylenediaminetetraacetic acid, or ethylenebis(oxyethylenenitrilo)tetraacetic acid in a buffer).

In some embodiments, the nanoemulsion further comprises an emulsifying agent to aid in the formation of the emulsion. Emulsifying agents include compounds that aggregate at the oil/water interface to form a kind of continuous membrane that prevents direct contact between two adjacent droplets. Certain embodiments of the present invention feature oil-in-water emulsion compositions that may readily be diluted with water to a desired concentration without impairing their anti-pathogenic properties.

In addition to discrete oil droplets dispersed in an aqueous phase, certain oil-in-water emulsions can also contain other lipid structures, such as small lipid vesicles (e.g., lipid spheres that often consist of several substantially concentric lipid bilayers separated from each other by layers of aqueous phase), micelles (e.g., amphiphilic molecules in small clusters of 50-200 molecules arranged so that the polar head groups face outward toward the aqueous phase and the apolar tails are sequestered inward away from the aqueous phase), or lamellar phases (lipid dispersions in which each particle consists of parallel amphiphilic bilayers separated by thin films of water).

These lipid structures are formed as a result of hydrophobic forces that drive apolar residues (e.g., long hydrocarbon chains) away from water. The above lipid preparations can generally be described as surfactant lipid preparations (SLPs). SLPs are minimally toxic to mucous membranes and are believed to be metabolized within the small intestine (see e.g., Hamouda et al., (1998) J. Infect. Disease 180: 1939).

In certain embodiments the emulsion comprises a discontinuous oil phase distributed in an aqueous phase, a first component comprising an alcohol and/or glycerol, and a second component comprising a surfactant or a halogen-containing compound. The aqueous phase can comprise any type of aqueous phase including, but not limited to, water (e.g., dionized water, distilled water, tap water) and solutions (e.g., phosphate buffered saline solution, or other buffer systems). The oil phase can comprise any type of oil including, but not limited to, plant oils (e.g., soybean oil, avocado oil, flaxseed oil, coconut oil, cottonseed oil, squalene oil, olive oil, canola oil, corn oil, rapeseed oil, safflower oil, and sunflower oil), animal oils (e.g., fish oil), flavor oil, water insoluble vitamins, mineral oil, and motor oil. In certain embodiments, the oil phase comprises 30-90 vol % of the oil-in-water emulsion (i.e., constitutes 30-90% of the total volume of the final emulsion), more preferably 50-80%. The formulations need not be limited to particular surfactants, however in certain embodiments, the surfactant is a polysorbate surfactant (e.g., TWEEN 20®, TWEEN 40®, TWEEN 60®, and TWEEN 80®), a pheoxypolyethoxyethanol (e.g., TRITON® X-100, X-301, X-165, X-102, and X-200, and TYLOXAPOL®), or sodium dodecyl sulfate, and the like.

In certain embodiments a halogen-containing component is present. the nature of the halogen-containing compound, in some preferred embodiments the halogen-containing compound comprises a chloride salt (e.g., NaCl, KCl, etc.), a cetylpyridinium halide, a cetyltrimethylammonium halide, a cetyldimethylethylammonium halide, a cetyldimethylbenzylammonium halide, a cetyltributylphosphonium halide, dodecyltrimethylammonium halides, tetradecyltrimethylammonium halides, cetylpyridinium chloride, cetyltrimethylammonium chloride, cetylbenzyldimethylammonium chloride, cetylpyridinium bromide, cetyltrimethylammonium bromide, cetyldimethylethylammonium bromide, cetyltributylphosphonium bromide, dodecyltrimethylammonium bromide, tetradecyltrimethylammonium bromide, and the like

In certain embodiments the emulsion comprises a quaternary ammonium compound. Quaternary ammonium compounds include, but are not limited to, N-alkyldimethyl benzyl ammonium saccharinate, 1,3,5-Triazine-1,3,5(2H,4H,6H)-triethanol; 1-Decanaminium, N-decyl-N,N-dimethyl-, chloride (or) Didecyl dimethyl ammonium chloride; 2-(2-(p-(Diisobuyl)cresosxy)ethoxy)ethyl dimethyl benzyl ammonium chloride; 2-(2-(p-(Diisobutyl)phenoxy)ethoxy)ethyl dimethyl benzyl ammonium chloride; alkyl 1 or 3 benzyl-1-(2-hydroxyethyl)-2-imidazolinium chloride; alkyl bis(2-hydroxyethyl)benzyl ammonium chloride; alkyl demethyl benzyl ammonium chloride; alkyl dimethyl 3,4-dichlorobenzyl ammonium chloride (100% C12); alkyl dimethyl 3,4-dichlorobenzyl ammonium chloride (50% C14, 40% C12, 10% C16); alkyl dimethyl 3,4-dichlorobenzyl ammonium chloride (55% C14, 23% C12, 20% C16); alkyl dimethyl benzyl ammonium chloride; alkyl dimethyl benzyl ammonium chloride (100% C14); alkyl dimethyl benzyl ammonium chloride (100% C16); alkyl dimethyl benzyl ammonium chloride (41% C14, 28% C12); alkyl dimethyl benzyl ammonium chloride (47% C12, 18% C14); alkyl dimethyl benzyl ammonium chloride (55% C16, 20% C14); alkyl dimethyl benzyl ammonium chloride (58% C14, 28% C16); alkyl dimethyl benzyl ammonium chloride (60% C14, 25% C12); alkyl dimethyl benzyl ammonium chloride (61% C11, 23% C14); alkyl dimethyl benzyl ammonium chloride (61% C12, 23% C14); alkyl dimethyl benzyl ammonium chloride (65% C12, 25% C14); alkyl dimethyl benzyl ammonium chloride (67% C12, 24% C14); alkyl dimethyl benzyl ammonium chloride (67% C12, 25% C14); alkyl dimethyl benzyl ammonium chloride (90% C14, 5% C12); alkyl dimethyl benzyl ammonium chloride (93% C14, 4% C12); alkyl dimethyl benzyl ammonium chloride (95% C16, 5% C18); alkyl dimethyl benzyl ammonium chloride (and) didecyl dimethyl ammonium chloride; alkyl dimethyl benzyl ammonium chloride (as in fatty acids); alkyl dimethyl benzyl ammonium chloride (C12-C16); alkyl dimethyl benzyl ammonium chloride (C12-C18); alkyl dimethyl benzyl and dialkyl dimethyl ammonium chloride; alkyl dimethyl dimethylbenzyl ammonium chloride; alkyl dimethyl ethyl ammonium bromide (90% C14, 5% C16, 5% C12); alkyl dimethyl ethyl ammonium bromide (mixed alkyl and alkenyl groups as in the fatty acids of soybean oil); alkyl dimethyl ethylbenzyl ammonium chloride; alkyl dimethyl ethylbenzyl ammonium chloride (60% C14); alkyl dimethyl isopropylbenzyl ammonium chloride (50% C12, 30% C14, 17% C16, 3% C18); alkyl trimethyl ammonium chloride (58% C18, 40% C16, 1% C14, 1% C12); alkyl trimethyl ammonium chloride (90% C18, 10% C16); alkyldimethyl(ethylbenzyl) ammonium chloride (C12-18); Di-(C8-10)-alkyl dimethyl ammonium chlorides; dialkyl dimethyl ammonium chloride; dialkyl dimethyl ammonium chloride; dialkyl dimethyl ammonium chloride; dialkyl methyl benzyl ammonium chloride; didecyl dimethyl ammonium chloride; diisodecyl dimethyl ammonium chloride; dioctyl dimethyl ammonium chloride; dodecyl bis(2-hydroxyethyl) octyl hydrogen ammonium chloride; dodecyl dimethyl benzyl ammonium chloride; dodecylcarbamoyl methyl dimethyl benzyl ammonium chloride; heptadecyl hydroxyethylimidazolinium chloride; hexahydro-1,3,5-thris(2-hydroxyethyl)-s-triazine; myristalkonium chloride (and) Quaternium 14; N,N-dimethyl-2-hydroxypropylammonium chloride polymer; n-alkyl dimethyl benzyl ammonium chloride; n-alkyl dimethyl ethylbenzyl ammonium chloride; n-tetradecyl dimethyl benzyl ammonium chloride monohydrate; octyl decyl dimethyl ammonium chloride; octyl dodecyl dimethyl ammonium chloride; octyphenoxyethoxyethyl dimethyl benzyl ammonium chloride; oxydiethylenebis (alkyl dimethyl ammonium chloride); quaternary ammonium compounds, dicoco alkyldimethyl, chloride; trimethoxysily propyl dimethyl octadecyl ammonium chloride; trimethoxysilyl quats, trimethyl dodecylbenzyl ammonium chloride; n-dodecyl dimethyl ethylbenzyl ammonium chloride; n-hexadecyl dimethyl benzyl ammonium chloride; n-tetradecyl dimethyl benzyl ammonium chloride; n-tetradecyl dimethyl ethylbenzyl ammonium chloride; and n-octadecyl dimethyl benzyl ammonium chloride.

Nanoemulsion formulations and methods of making such are well known to those of skill in the art and described for example in U.S. Pat. Nos. 7,476,393, 7,468,402, 7,314,624, 6,998,426, 6,902,737, 6,689,371, 6,541,018, 6,464,990, 6,461,625, 6,419,946, 6,413,527, 6,375,960, 6,335,022, 6,274,150, 6,120,778, 6,039,936, 5,925,341, 5,753,241, 5,698,219, an d5,152,923 and in Fanun et al. (2009) Microemulsions: Properties and Applications (Surfactant Science), CRC Press, Boca Ratan Fla.

In certain embodiments, one or more active agents described herein can be provided as a “concentrate”, e.g., in a storage container (e.g., in a premeasured volume) ready for dilution, or in a soluble capsule ready for addition to a volume of water, alcohol, hydrogen peroxide, or other diluent.

In certain embodiments, the rapamycin analogs described herein (e.g., compound 390, compound 405, compound 384, and the like) are formulated as inclusion complexes. While not limited to cyclodextrin inclusion complexes, it is noted that cyclodextrin is the agent most frequently used to form pharmaceutical inclusion complexes. Cyclodextrins (CD) are cyclic oligomers of glucose, that typically contain 6, 7, or 8 glucose monomers joined by α-1,4 linkages. These oligomers are commonly called α-CD, β-CD, and γ-CD, respectively. Higher oligomers containing up to 12 glucose monomers are known, and contemplated to in the formulations described herein. Functionalized cyclodextrin inclusion complexes are also contemplated. Illustrative, but non-limiting functionalized cyclodextrins include, but are not limited to sulfonates, sulfonates and sulfinates, or disulfonates of hydroxybutenyl cyclodextrin; sulfonates, sulfonates and sulfinates, or disulfonates of mixed ethers of cyclodextrins where at least one of the ether substituents is hydroxybutenyl cyclodextrin. Illustrative cyclodextrins include a polysaccharide ether which comprises at least one 2-hydroxybutenyl substituent, wherein the at least one hydroxybutenyl substituent is sulfonated and sulfinated, or disulfonated, and an alkylpolyglycoside ether which comprises at least one 2-hydroxybutenyl substituent, wherein the at least one hydroxybutenyl substituent is sulfonated and sulfinated, or disulfonated. In various embodiments inclusion complexes formed between sulfonated hydroxybutenyl cyclodextrins and one or more of the active agent(s) described herein are contemplated. Methods of preparing cyclodextrins, and cyclodextrin inclusion complexes are found for example in U.S. Patent Publication No: 2004/0054164 and the references cited therein and in U.S. Patent Publication No: 2011/0218173 and the references cited therein.

In certain embodiments the rapamycin analogs described herein can also be administered using medical devices known in the art. For example, in one embodiment, a pharmaceutical composition of the invention can be administered with a needleless hypodermic injection device, such as the devices disclosed in U.S. Pat. No. 5,399,163; U.S. Pat. No. 5,383,851; U.S. Pat. No. 5,312,335; U.S. Pat. No. 5,064,413; U.S. Pat. No. 4,941,880; U.S. Pat. No. 4,790,824; or U.S. Pat. No. 4,596,556. Examples of well-known implants and modules useful for such deliver include, but are not limited to U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. In a specific embodiment a rapamycin analogue may be administered using a drug-eluting stent, for example one corresponding to those described in WO 01/87263 and related publications or those described by Perin (Perin, E C, 2005). Many other such implants, delivery systems, and modules are known to those skilled in the art.

The dosage to be administered of a rapamycin analog described herein will vary according to the particular compound, the disease involved, the subject, and the nature and severity of the disease and the physical condition of the subject, and the selected route of administration. The appropriate dosage can be readily determined by a person skilled in the art. For example, without limitation, a dose of up to 15 mg daily e.g. 0.1 to 15 mg daily (or a higher dose given less frequently) may be contemplated.

In certain embodiments the compositions may contain from 0.1%, e.g. from 0.1-70%, or from 5-60%, or preferably from 10-30%, of one or more rapamycin analogs, depending on the method of administration.

It will be recognized by one of skill in the art that the optimal quantity and spacing of individual dosages of a rapamycin analog described herein will be determined by the nature and extent of the condition being treated, the form, route and site of administration, and the age and condition of the particular subject being treated, and that a physician will ultimately determine appropriate dosages to be used. This dosage may be repeated as often as appropriate. If side effects develop the amount and/or frequency of the dosage can be altered or reduced, in accordance with normal clinical practice.

### EXAMPLES

The following examples are offered to illustrate, but not to limit the claimed invention.

### Example 1

### Isolation of 27-O-Desmethyl-39-Desmethoxy Rapamycin

Methods of production and isolation were carried out as generally as described in PCT Publication No: WO 2004/007709 (PCT/GB2003/003230). The S. rapamycinicus strain MG2-10 (see WO 2004/007709) was conjugated with pLSS227, containing rapJ, rapM, rapN, rapO and rapLhis in pSGSet1. This plasmid was constructed as described in WO 2004/007709. The strain was grown as follows:

### Fermentation of 27-O-Desmethyl-39-Desmethoxy Rapamycin

TABLE 2
RapV7 seed medium recipe.

| Ingredient | Manufacturer | Quantity | |
|---|---|---|---|
| Corn steep solids | | 4.0 | g |
| Soy flour (Toasted Nutrisoy) | ADM | 5.0 | g |
| White dextrin WBD | Avedex | 35.0 | g |
| Ammonium sulphate | Reidel de Hahn | 2.0 | g |
| Lactic acid | Fluka | 1.6 | ml |
| Calcium carbonate | Sigma | 7 | g |
| Tap H₂O to final vol. of | | 1.0 | L |

Seed Culture Preparation

2000 ml Erlenmeyer flasks were filled with 400 ml RapV7 seed medium and sterilized by autoclave (121° C.; 30 min). A frozen (−80° C.) spore stock of S. rapamycinicus MG2-10 [pLSS227] was fully thawed and a 0.05% inoculum added to 400 ml sterile RapV7 seed medium which was pre-warmed and oxygenated at 28° C., 250 rpm, 2.5 cm throw for 30 min. This was incubated at 28° C., 250 rpm, 2.5 cm throw for 48 hrs.

Production Medium Recipe

TABLE 3
MD6 Production Medium

| Ingredient | Manufacturer | Quantity | |
|---|---|---|---|
| Soy flour (Toasted Nutrisoy) | ADM | 30 | g |
| Corn Starch | Sigma | 30 | g |
| White dextrin WBD | Avedex | 19 | g |
| Whole yeast | Fermipan | 3 | g |
| Corn steep solids | Roquette | 1 | g |
| K₂HPO₄ | Sigma | 2.5 | g |
| KH₂PO₄ | Sigma | 2.5 | g |
| Ammonium sulphate | Reidel de Haen | 10 | g |
| Sodium chloride | Fisher | 5 | g |
| Calcium carbonate | Sigma | 10 | g |
| MnCl₂•4H₂O | | 0.01 | g |
| MgSO₄•7H₂O | | 0.0025 | g |
| FeSO₄•7H₂O | Sigma | 0.12 | g |
| ZnSO₄•7H₂O | | 0.05 | g |
| MES | Acros Organics | 21.2 | g |
| α-Amylase | Sigma | 0.4 | ml |
| SAG471 (antifoam) | GE | 0.5 | ml |

| | |
|---|---|
| Tap water | to 1 L |

pH should be 6.0-7.0. Adjust prior to sterilization, if necessary
Sterilization by autoclave (121° C.; 30 min)

TABLE 4
Post-sterilization additions to MD6 base medium (filter sterilized)

| Ingredient | Quantity | |
|---|---|---|
| 40% D-fructose | 50 | ml/L |
| 10% L-lysine | 20 | ml/L |
| (monohydrochloride) | | |

Trans-4-hydroxy cyclohexane carboxylic acid was prepared 24 hours in advance in MeOH; final concentration 2 mM. 15 liters base medium (MD6 production medium without fructose or L-lysine) was transferred to a V7 Braun 22 L fermenter and sterilized. Following autoclaving, pre-sterilized fructose (15 g/L) and L-lysine (0.5 g/L) were added. The entire seed culture (400 ml) was transferred to production media in the fermentation vessel. Starting parameters were T=26° C., 7.5 L/min air, 200 rpm, Aeration rate was: 0.5 v/v/m, automatic pH control to pH set point 6.5 (6.4-6.6), pH controlled with 15% NaOH. Dissolved oxygen was controlled with agitation cascade at 30% air saturation. Trans-4-hydroxy cyclohexane carboxylic acid in MeOH was added at 24 hours of fermentation to final concentration of 2 mM. SAG 471 (0.5 ml/L) used to prevent extensive foaming. The bioprocess was continued for 6 days.

### Preparation of Crude Extract of 27-O-Desmethyl-39-Desmethoxy Rapamycin

The whole broth was centrifuged at 3500 rpm (RCF 3300 g), 25 min. Clarified broth was assayed and discarded if less than 5% target compound detected. Cell pellet was removed from centrifuge pots with acetonitrile and decanted into 10 L duran. Further acetonitrile was added to give solvent to cell volume ration of 2:1; mixture stirred with overhead electric paddle stirrer, 600 rpm, 1 hour. Following stirring, the mixture was left to settle under gravity for 15 min. The solvent/aqueous layer was removed as extract 1. A further 2 volumes of acetonitrile were added to remaining cells; the mixture stirred and allowed to settle again, as above, to obtain extract 2. Any remaining 27-O-desmethyl-39-desmethoxy rapamycin in cell pellet was removed by third extraction, if required.

Extracts from cell biomass were concentrated in vacuo to residual aqueous extract. The aqueous fraction was extracted into an equal volume of ethyl acetate. The Ethyl acetate extract was concentrated in vacuo to yield an oily crude extract. This was dissolved in 80% MeOH in water and mixed with 1 volume hexane. The hexane partition was discarded and solvent removed in vacuo to yield final crude extract.

### Silica Chromatography

The crude extract was dissolved in methanol and a quantity of silica gel approximately equal to that of the extract added. Solvent was removed in vacuo to yield a free-flowing powder. Impregnated silica was loaded onto a silica gel column (20×5 cm) and eluted with 100% CHCl₃. Polarity was gradually increased by addition of MeOH to maximum of 5% MeOH. Approximately 20×250 ml fractions were collected and monitored by HPLC. Fractions containing Any remaining 27-O-desmethyl-39-desmethoxy rapamycin were loaded onto second silica gel column (15×2 cm) and eluted with gradient of hexane and ethyl acetate, starting with 1 L 1:1 mixture, followed by 1 L 40:60 mixture and finally with 100% EtOAc. Approximately 20×250 ml fractions were collected and monitored by HPLC. Fractions containing any remaining 27-O-desmethyl-39-desmethoxy rapamycin were combined and the solvents removed in vacuo to yield semi pure compound.

### Final Purification by Preparative HPLC

Preparative HPLC was then performed using Waters X-Terra MS C18 column (OBD 10 μm; 19×250 mm) with security guard. The extract was dissolved in acetonitrile and 10 injections loaded onto column. Elution was in 55% to 80% acetonitrile in water gradient for 30 min. Any remaining fractions containing pure 27-O-desmethyl-39-desmethoxy rapamycin were pooled and solvent removed in vacuo.

### Analysis of 27-O-Desmethyl-39-Desmethoxy Rapamycin by HPLC

Detection of 27-O-desmethyl-39-desmethoxy rapamycin was carried out using a Phenomenex Gemini-NX C18 3u 110A reversed-phase column (150×4.6 mm, 3 μm particle size) with security guard cartridge containing same silica as column. HPLC was conducted as follows:

System 1: Mobile phase A: water:acetonitrile (9:1) containing 0.01 M ammonium acetate and 0.1% TFA. Mobile phase B: water acetonitrile (1:9) containing 0.01 M ammonium acetate and 0.1% TFA; RT 9.7 min;

System 2: Mobile phase A: water+0.1% formic acid. Mobile phase B: acetonitrile+0.1% formic acid; RT 8.2 min;

Flow rate: 1 ml/min;

Column oven temperature: 50 C;

λmax: 280 nm;

Gradient: T=0 min, 55% B; T=10 min, 95% B; T=12 min, 95% B; T=12.5 min, 55% B; T=15 min, 55 min.

### Example 2

In Vitro Assessment of mTORC1/2 Selectivity Data on Various Compounds—Including Compound 390

### Method.

PC3 cells were maintained in F12K media supplemented with 10% FBS, 1% Penicillin/Streptomycin, and 2 mM L-Glutamine and cultured at 37° C. under an atmosphere of 95% air and 5% CO₂. Cells were treated with 100 nM Rapamycin or rapalogs described herein (e.g., compound 390) and harvested in RIPA buffer (300 mM NaCl, 1.0% NP-40, 0.5% sodium deoxycholate, 0.1% SDS, 50 mM Tris (pH 8.0), protease inhibitor cocktail (Roche), phosphatase inhibitor 2, 3 (Sigma). Protein concentrations were determined using the DC protein assay (Biorad). Equal amounts of protein were resolved by SDS-PAGE and transferred to nitrocellulose membrane using the Nu-Page system. The membranes were blocked for 1 h in 5% milk and incubated overnight in the appropriate antibodies. The following day, blots were washed 3 times in TBST, incubated for 2 h with secondary antibodies, and finally washed an additional 3 times in TBST.

### Results.

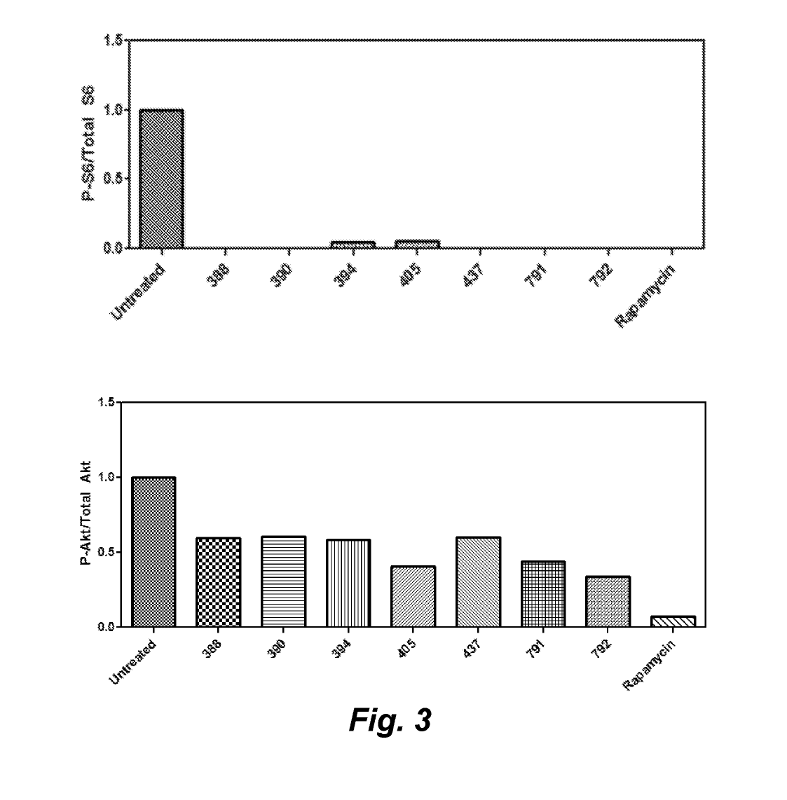

Compounds 388, 390, 394, 405, 437, 791 and 792 exhibited full mTORC1 inhibition at 100 nM yet they displayed partial inhibitory action on mTORC2 to 50-75% of activity compared to rapamycin, a dual mTOR inhibitor. This includes compounds nID 388, 390, 394, 405, 437, 791 and 792 (see, e.g., FIG. 3).

### Example 3

### In Vitro Determination of Relative mTORC1/mTORC2 SELECTIVITY OF mTORC1-Selective Rapalogs—Including Compound 390

### Method.

PC3 cells were maintained in F12K media (ATCC/GIBCO, Cat# ATCC 30-2004) supplemented with additional 10% FBS (Gemini, cat#100-106), 1% Penicillin/Streptomycin (Life Technologies, cat #15140-122), and 2 mM L-Glutamine (Life Technologies, cat#25030) and cultured at 37° C. For AlphaLISA experiments, cells were seeded in 96-well plates for 24 hours and treated at various concentrations of compound (from approx. 8 fM to 10 μM) for 24 hours. Cells were harvested by lysis in the buffer supplied with the AlphaLISA kit.

mTORC1 inhibition was determined using the AlphaLISA® SureFire® kit (Perkin Elmer) which measures phosphorylation of S6 kinase at positions Ser²⁴⁰ and Ser²⁴⁴. mTORC2 inhibition was determined by the AlphaLISA® SureFire® kit for Akt 1/2/3, which determines phospholyltation of Akt protein at position Ser⁴⁷³. Cells from plates were lysed and incubated for 10 min at room temperature while shaking. Cell lysates were incubated with acceptor mix for 2 hours at room temperature; the donor mix was then added and the resulting solution was incubated for 2 hours at room temperature. AlphaLISA® signal was determined on a Fusion-Alpha FP HT (Perkin Elmer). Percent inhibition was calculated by comparison to the highest inhibition value obtained in the response-concentration curve. IC₅₀s were calculated using Prism software. All IC₅₀ experiments were conducted in triplicates and in all cases against rapamycin and vehicle controls.

### Results.

The results validate the low mTORC2 inhibitory activity of nID390, as was also confirmed in the Western blot analysis: nID390 exhibits a nearly 150-fold lower inhibition of mTORC2 compared to rapamycin. This quantitative now information explains well the vastly improved safety profile of the compound over rapamycin, even when administered at 1.5-fold higher dose in vivo (as will be demonstrated in the next set of results). We analyzed the mTORC1 selectivity of nID390 vs. rapamycin by constructing the selectivity ratios presented in Table 2, which were determined by dividing the mTORC2 IC₅₀ for each measurement by the average mTORC1 IC₅₀ for each compound. Using this definition, higher IC₅₀^{mTORC2}/IC₅₀^{mTORC1} ratio indicates higher selectivity for mTORC1 inhibition relative to mTORC2 inhibition. As indicated in Table 5, compound 390 exhibits an average 51-fold increased selectivity for mTORC1 over mTORC2 inhibition (range 52-63) as compared to rapamycin.

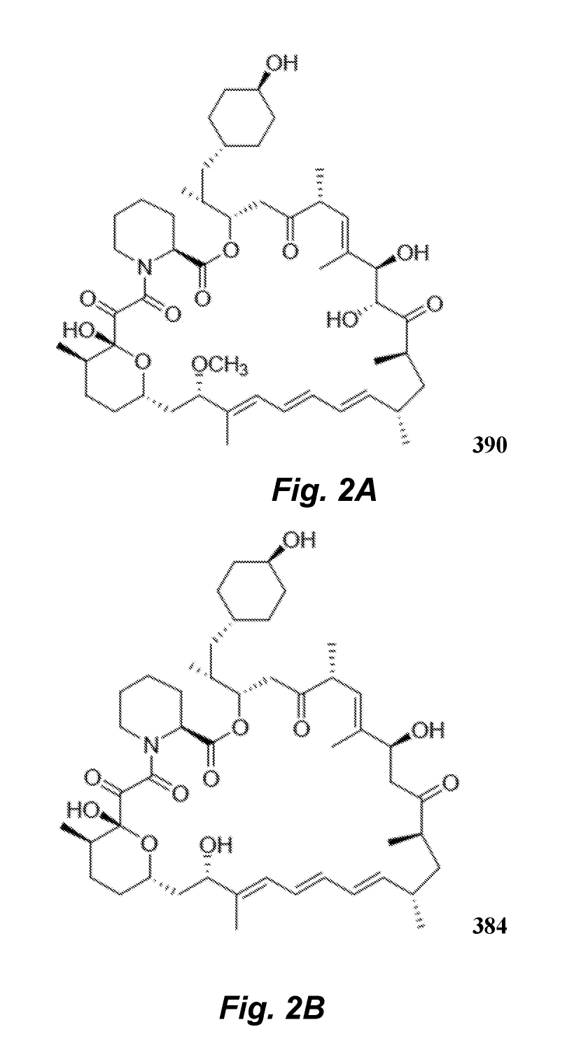

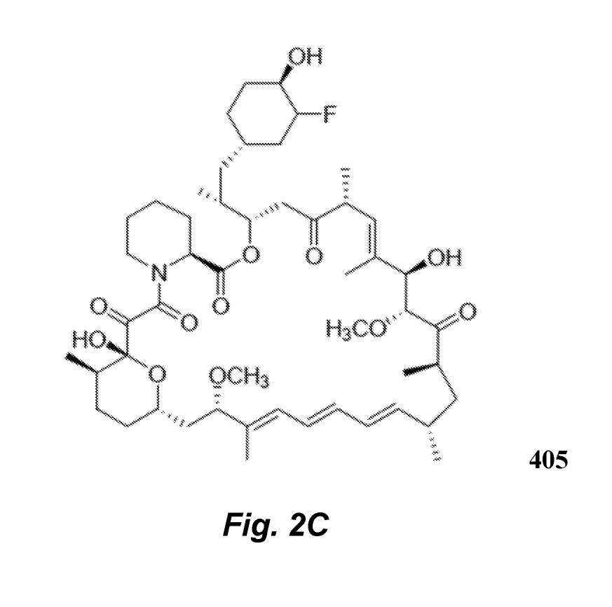

We further determined the IC₅₀ of mTORC1 and mTORC2 inhibition for two other rapalog compounds described herein. Compounds 384 and 405 (see, FIGS. 2B and 2C). The compounds were selected on the basis of both their Western blot IC₅₀ results but also the interesting in vivo findings. The IC₅₀s were determined in four different measurements, each of which consisted of three replicates (N=12). As the selectivity analysis results (given in Table 5) suggest, compound 384 exhibits on average nearly 1000-fold selectivity for mTORC1 over mTORC2 inhibition; this represents a nearly 110-fold improvement compared to rapamycin. Further, and despite its stronger mTORC2 inhibitory activity compared to the other compounds, compound 405 exhibits 105-fold selectivity for mTORC1 inhibition over mTORC2; this represents a 12.5-fold improvement over rapamycin (range: 0.85-15).

TABLE 5
mTORC1 Selectivity ratios for rapamycin
and compound 390. mTORC1 selectivity was defined as
the ratio IC₅₀^{mTORC2}/IC₅₀^{mTORC1}, as obtained
in the above studies.

| | | | Average | |
|---|---|---|---|---|
| | Replicate | IC50^{mTORC2}/ | IC50^{mTORC2}/ | Std. |
| Compound | (N = 3) | IC50^{mTORC1} | IC50^{mTORC1} | Error |
| Rapamycin | #1 | 10.74 | 8.44 | 1.17 |
| | #2 | 10.04 | | |
| | #3 | 2.37 | | |
| | #4 | 10/64 | | |
| nID390 | #1 | 477.07 | 429.90 | 33.81 |
| | #2 | 533.79 | | |
| | #3 | 445.85 | | |
| | #4 | 262.89 | | |
| nID384 | #1 | 1263.5 | 926.3 | 80.5 |
| | #2 | 602.6 | | |
| | #3 | 833.2 | | |
| | #4 | 1005.7 | | |
| mID405 | #1 | 127.4 | 38.73 | 8.25 |
| | #2 | 7.2 | | |
| | #3 | 174.8 | | |
| | #4 | 111.7 | | |

### Example 4

### Comparison of mTORC1/2 Inhibition of mTORC1-Selective Compounds to Known Dual mTOR Inhibitors (Rapamycin, Everolimus, Temsirolimus)—Including nID390 (In Vitro)

### Method.

PC3 cells were maintained in F12K media supplemented with 10% FBS, 1% Penicillin/Streptomycin, and 2 mM L-Glutamine and cultured at 37° C. Cells were treated with 100 nM Rapamycin, Temsirolimus, Everolimus, and rapalog compounds 390, 394, 824, 384 or 405 for 24 h and harvested in RIPA buffer (300 mM NaCl, 1.0% NP-40, 0.5% sodium deoxycholate, 0.1% SDS, 50 mM Tris pH 8.0. Protein concentrations were determined using the DC protein assay (Biorad).

Equal amounts of protein were resolved by SDS-PAGE and transferred to nitrocellulose membrane using the Invitrogen Nu-Page system. The membranes were blocked for 1 h in 5% milk and incubated overnight in the appropriate antibodies. The following day, blots were washed 3 times in TBST, incubated for 2 h with secondary antibodies (donkey anti rabbit hrp conjugated), and finally washed an additional 3 times in TBST. ECL Prime (Amersham) was used to detect the proteins of interest and blots were quantified using ImageJ software.

### Results.

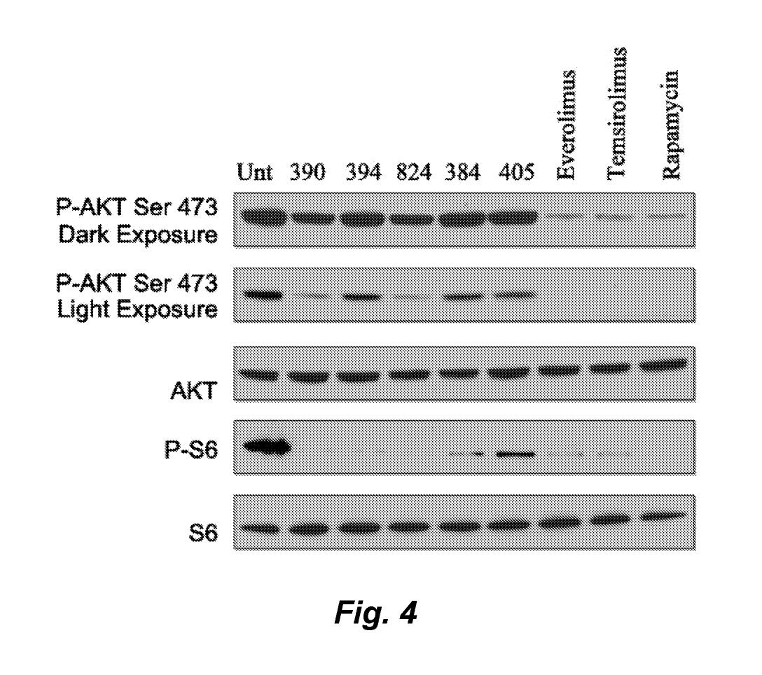

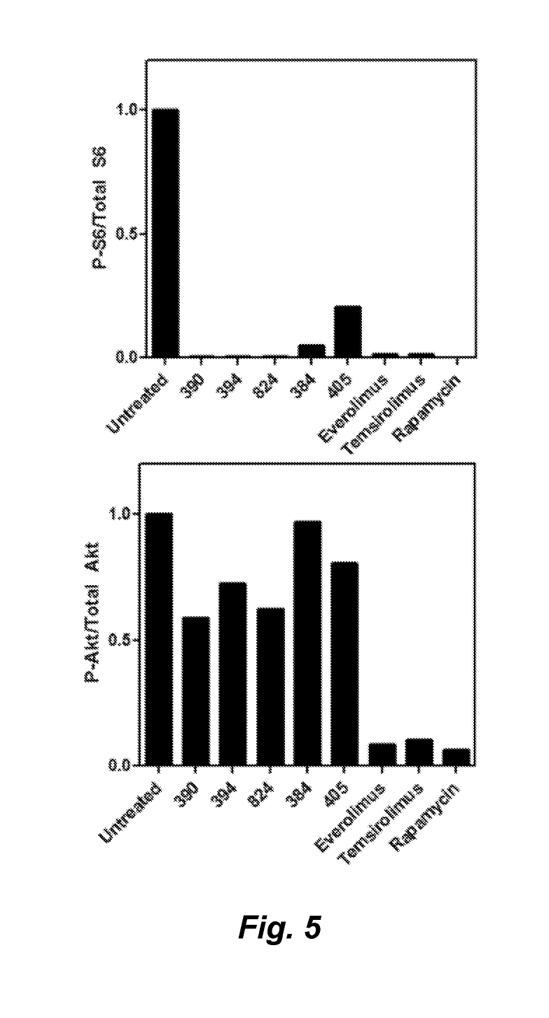

Everolimus and Temsirolimus performed identically to rapamycin with respect to both mTORC1 (p-S6K) and mTORC2 (p-Akt) inhibition, reducing mTORC2 activity to approximately 5% of control. Most of the new rapalogs described herein completely inhibited p-S6K activity, similarly to rapamycin. The compounds retained nearly full mTORC1 activity yet exhibited diminished mTORC2 inhibitory activity, less than 50% of the controls. In particular, compound nID384 appears to be a very selective mTORC1 inhibitor, as it exhibited near total inhibition of mTORC1 while it did not appear to exert any effect ton mTORC2 activity (see, e.g., FIGS. 4 and 5).

### Example 5

### In Vivo Assessment of mTORC1 Selectivity of Various Compounds—Including nID390 (In Vivo)

### Purpose.

Recent evidence has revealed that inhibition mTORC2 by rapamycin is uncoupled from its effects on lifespan and is responsible for several of the adverse effects of the compound, such as glucose intolerance, impaired insulin sensitivity, glucose homeostasis and lipid dysregulation (Lamming et al. (2012) 335(6076): 1638-1643), to name a few. This side effect profile seems to be related to the TORC2 inhibition of rapamycin in specific tissues in mice, namely liver, white adipose tissue and skeletal muscle (Id.).

This study was intended to
i) assess the mTORC1 selectivity of compound 390 compared to rapamycin (and vehicle control) in vivo and to
ii) evaluate the impact of compound 390 administration on the side effects associated with mTORC2 inhibition side effects (glucose levels, glucose tolerance, insulin resistance and lipid upregulation) in comparison to rapamycin and vehicle controls.

### Methods.

10-week old female C57BL/6J mice were given intraperitoneal injections of 8 mg/kg rapamycin (LC laboratories), 12 mg/kg compound 390 or vehicle every other day for three weeks (N=5 mice/group), such as to mimic chronic exposure conditions. 24 hours after the last injection, tissues were dissected from the mice and immediately frozen in liquid nitrogen. The tissues were homogenized using the Omni TH homogenizer (Omni International) on ice in RIPA buffer and then centrifuged at 13,000 rpm for 15 minutes at 4° C. The supernatants were collected and protein concentration was determined using the DC protein assay (Biorad).

The mTORC1/2 inhibition was determined by Western blots. Equal amounts of protein were resolved by SDS-PAGE and transferred to nitrocellulose membrane using the Invitrogen Nu-Page system. The membranes were blocked for 1 h in 5% milk and incubated overnight in the appropriate antibodies. The following day, blots were washed 3 times in TBST, incubated for 2 h with secondary antibodies (donkey anti rabbit hrp conjugated), and finally washed an additional 3 times in TBST. ECL Prime (Amersham) was used to detect the proteins of interest and ImageJ software was used to quantify the band intensity of the Western blots.

Steady state glucose levels were determined after one week from study initiation using a Bayer Contour blood glucose meter and test strips from small blood samples, which were withdrawn from a tail vein nick. At the end of the second week, the animals were challenged by administration of 2 mg/kg glucose; glucose levels were determined for two hours following the challenge at 30 min intervals.

Plasma lipid levels were determined at the end of the three-week exposure. Free fatty acids in the serum were measured by the HR Series NEFA-HR(2) (Wako Diagnostics, Richmond, Va.). Triglycerides and cholesterol in serum were measured by Triglycerides Liquicolor Test and Cholesterol Liquicolor Test (Stanbio laboratory, Boerne, Tex.), respectively.

### Results.

(a) mTORC1/2 Selectivity.

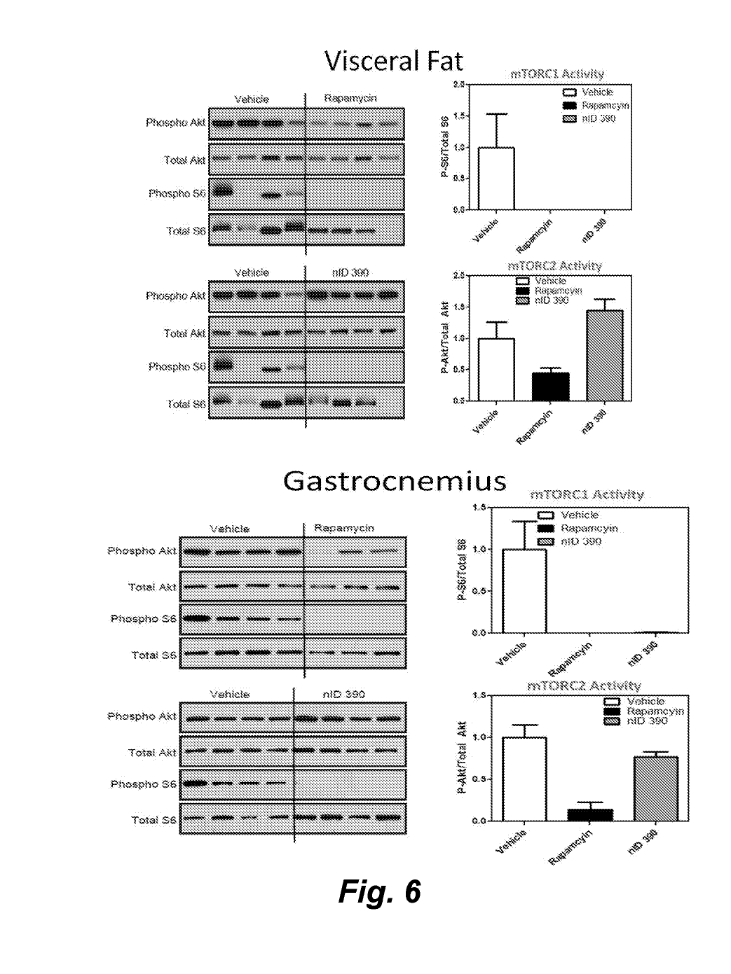

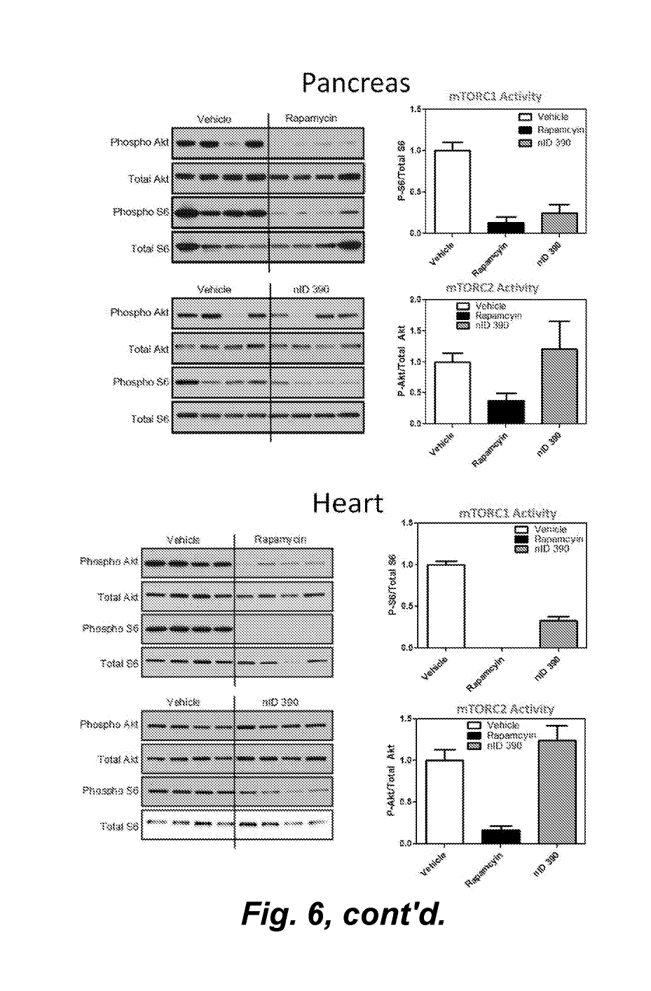

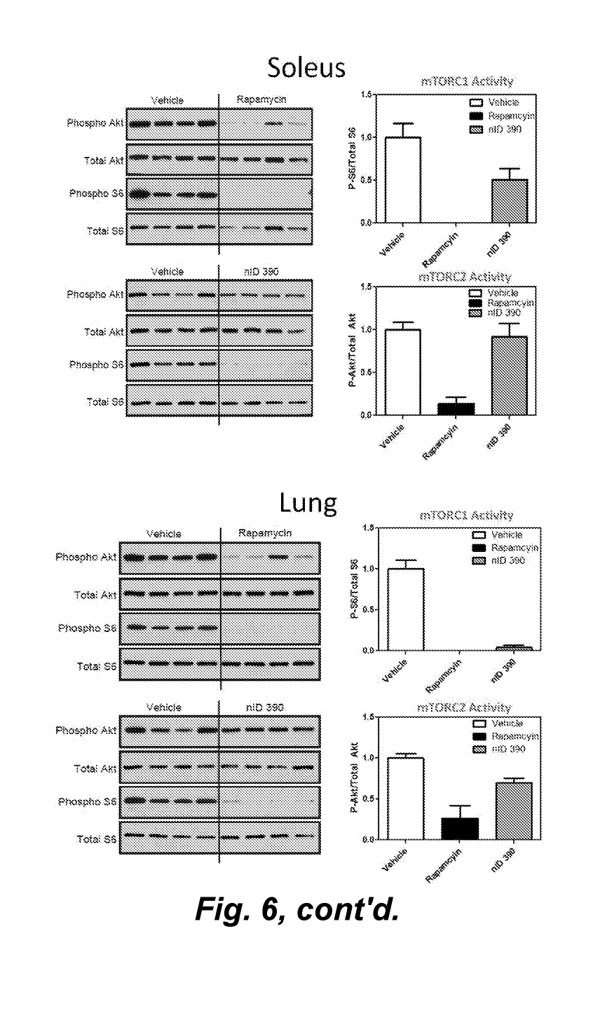

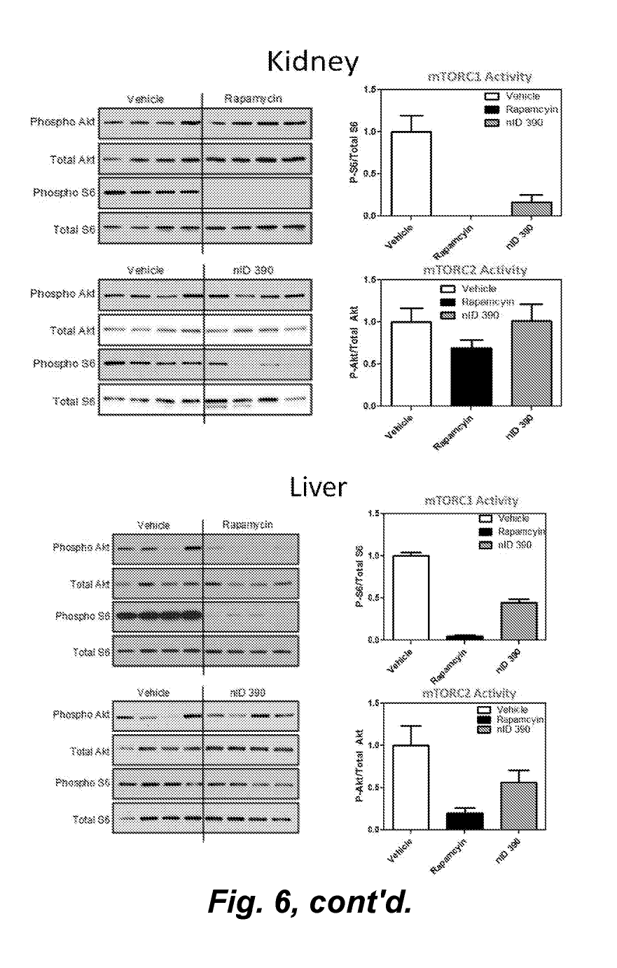

Rapamycin exhibits strong inhibition of mTORC1 in all tissues examined in this study; it also produces excellent inhibition of mTORC2 across all tissues with the exception of liver, for which inhibition of mTORC2 is only partial. Similarly, compound 390 exhibits strong inhibition of mTORC1 in visceral fat, gastrocnemius muscle, pancreas, lung and kidney while it only partially inhibits mTORC1 in the soleus muscle and the liver. However, compound 390 exhibits very weak (gastrocnemius muscle, soleus muscle, lung liver) or no inhibition of mTORC2 across all other tissues (heart, visceral fat, pancreas, soleus muscle, kidney) examined in this study. In fact, in the latter tissues, a small upregulation of mTORC2 activity is observed, an indication of high mTORC1 selectivity (see, e.g., FIG. 6).

(b) Lipid Regulation.

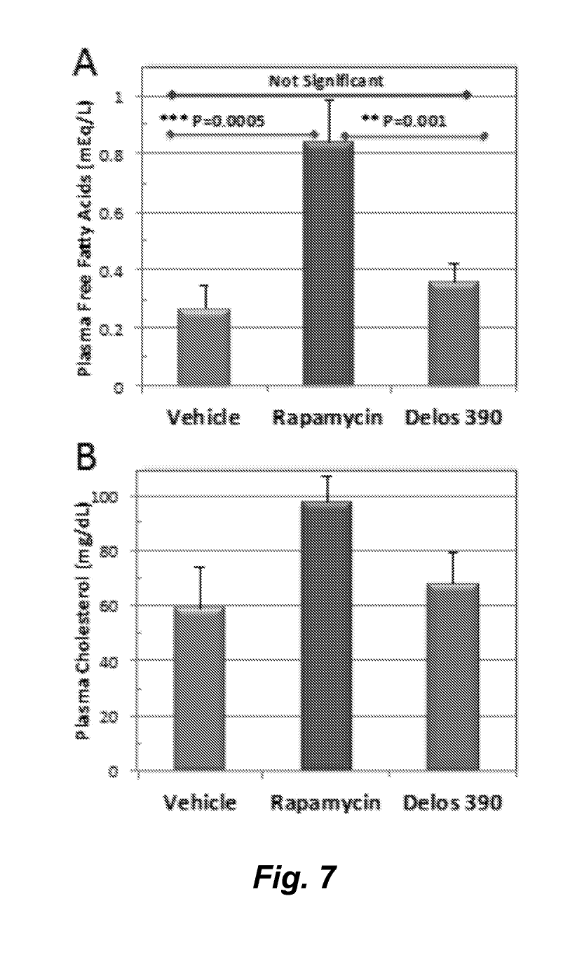

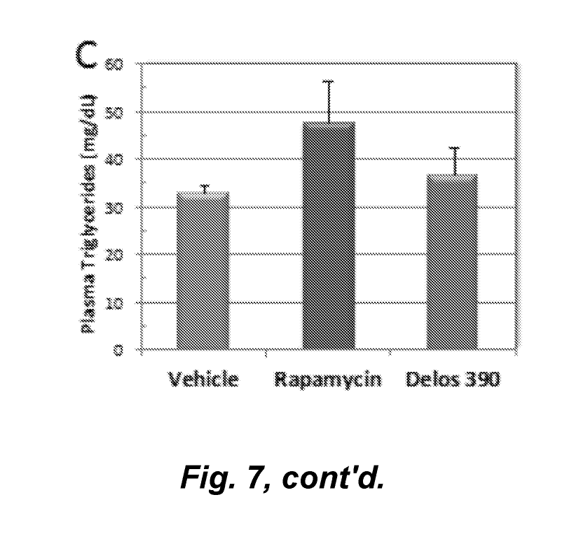

After 4 weeks of administration mice receiving rapamycin exhibited a substantial and statistically significant increase of the plasma free fatty acids. Similar increases, albeit not statistically significant were observed in the plasma levels of cholesterol and triglycerides. In contrast, administration of compound 390 even at nearly 50% higher dose of 12 mg/kg, did not produce any changes in the plasma levels of all these lipids, which are statistically the same as the vehicle-receiving animal controls (see, e.g., FIG. 7).

(c) Steady State Glucose.

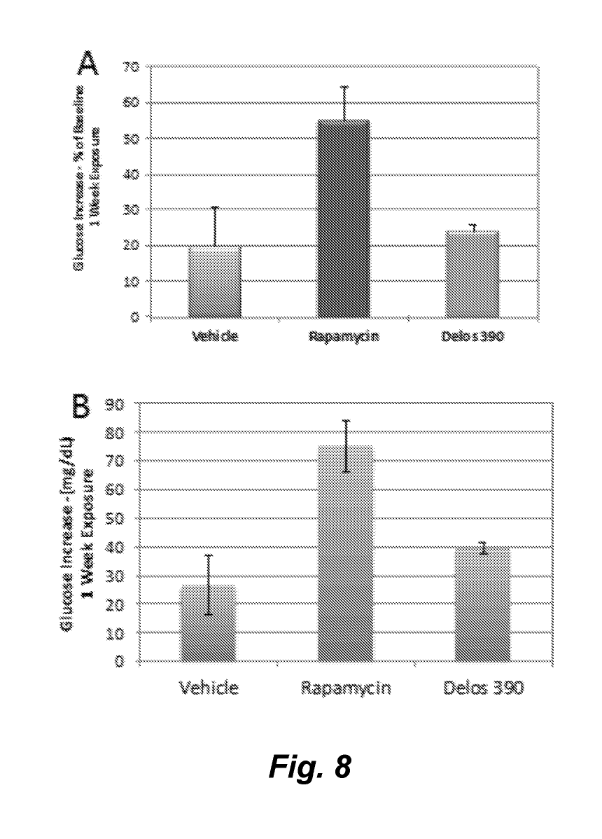

One of the most profound effects of rapamycin in vivo is the increase of the glucose steady state levels in vivo. As seen in FIGS. 8A and 8B, even after one week of exposure rapamycin administration of 8 mg/kg results in significant increases of the glucose steady state levels: from approx. 26 (in the vehicle-receiving animals) to 75 mg/dL for the rapamycin-receiving animals, representing a 3-fold increase (FIG. 8B) correlating to approx. 55% increase from baseline levels (FIG. 8A). In contrast, compound 390 administration does not produce any substantial or statistically significant increase of the steady state glucose plasma levels, which are nearly identical to the vehicle-receiving controls (see, e.g., FIG. 8).

(d) Glucose Intolerance.

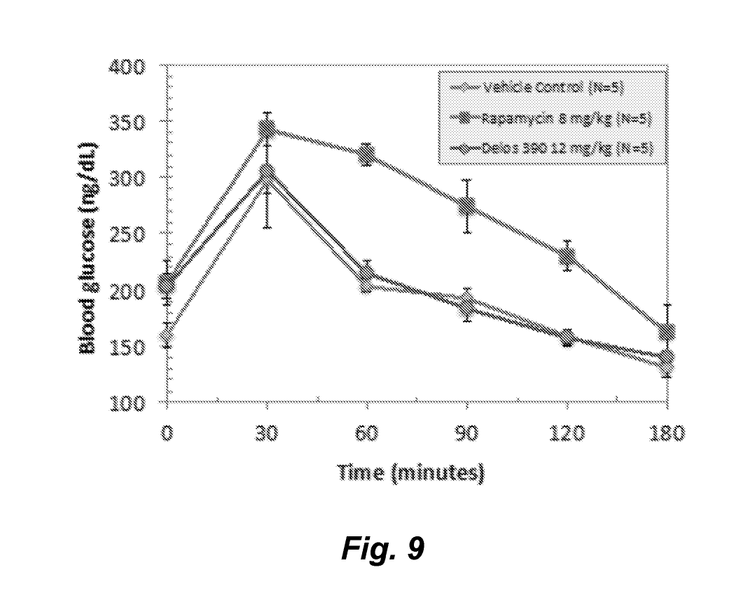

After a challenge of 2 mg/kg of glucose, animals receiving rapamycin for 1 week produce a glucose response that deviates significantly from the response observed with the control animals, which signifies changes in glucose homeostasis and intolerance to glucose challenge. In contrast, the animals receiving compound 390 for one week have a response that is indistinguishable from the vehicle-receiving control animals, thus suggesting that the compound 390 does not produce any glucose intolerance in vivo see, e.g., FIG. 9).

(e) Insulin Resistance.

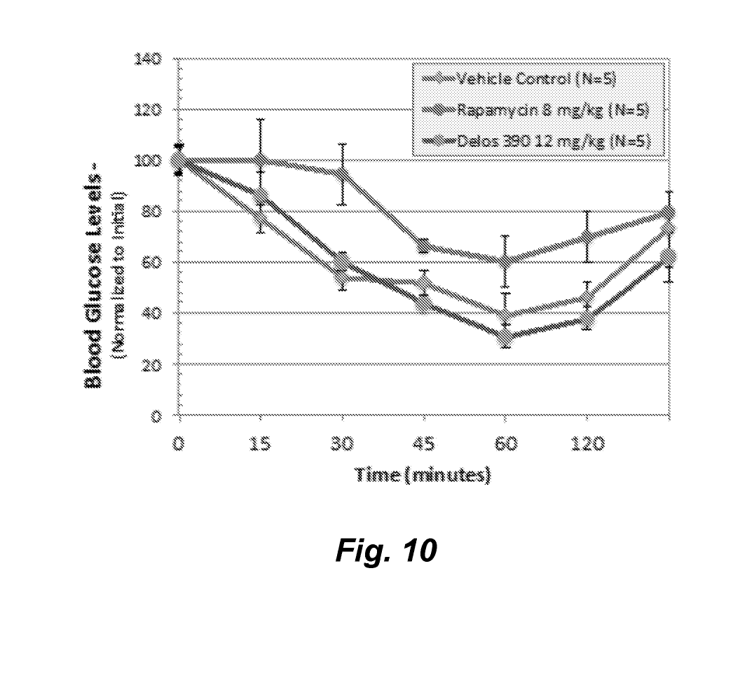

After a challenge of 0.75 IU/kg of insulin, animals receiving rapamycin for 2 weeks produce a glucose response that deviates significantly from the response observed with the control animals. This response is indicative of changes in insulin processing resembling insulin intolerance. In contrast, the animals receiving compound 390 for two weeks have a response that is indistinguishable from the vehicle-receiving control animals, thus suggesting that the compound 390 does not produce any changes in the processing of insulin in vivo (see, e.g., FIG. 10).

### Example 6

### In Vivo Assessment of mTORC2-Related Side Effects Following Chronic Administration of mTORC1-Selective Inhibitor Compound 390

### Purpose.

The objectives of this study were to further:
i) assess the mTORC1/2 inhibition of compound 390 compared to rapamycin (and vehicle control) in selected tissues in vivo (in male mice);
ii) evaluate the impact of compound 390 in comparison to rapamycin and vehicle controls on metabolism: glucose intolerance, pyruvate intolerance, fat mass; and
iii) evaluate the impact of rapamycin vs. compound 390 on the assembly of mTORC2 in selected tissues.

### Methods.

Fortyfive C57BL/6J male mice, housed three per cage, were weighed and their body composition was measured with an EchoMRI 3-in-1 system. Cages were sorted into three groups—vehicle, rapamycin, and compound 390—such that the average weight was similar for all three groups. Mice received every-other-day injections of either vehicle, rapamycin (8 mg/kg), or compound 390 (12 mg/kg). A glucose tolerance test was performed after 2 weeks (morning after the 8^{th} injection). A pyruvate tolerance test was performed after 3 weeks. Glucose stimulated insulin secretion assay was performed after 4 weeks and additional blood was collected for whole blood analysis. Body composition was measured on day −2 and day 31.

Glucose and Pyruvate Tolerance Tests and Glucose-Stimulated Insulin Tests

Mice were fasted overnight for 16 hours and then injected with either glucose (1 g/kg) or pyruvate (2 g/kg) intraperitoneally. For glucose and pyruvate tolerance tests, small blood samples were taken from a tail vein nick at time intervals and read using a Bayer Contour blood glucose meter and test strips. For glucose-stimulated insulin secretion, blood glucose levels were read using a glucometer and then 50 μL of blood was collected into a heparinized tube immediately prior to and 15 minutes following glucose administration. Insulin levels were determined using a Mouse Insulin ELISA kit (Crystal Chem).

Antibodies

For western blotting, antibodies to phospho-Akt S473 (4060), Akt (4691), phospho-S6 ribosomal protein (2215), S6 ribosomal protein (2217), phospho-4E-BP1 S65 (9451) and 4E-BP1 (9452) were from Cell Signaling Technology.

Immunoblotting

Cells and tissue samples were lysed in cold RIPA buffer supplemented with phosphatase inhibitor and protease inhibitor cocktail tablets (PI88669, Fisher Scientific). Tissues were lysed in RIPA buffer using a FastPrep 24 (M.P. Biomedicals) with bead-beating tubes and ceramic beads (Mo-Bio Laboratories), and then centrifuged. Protein concentration was determined by Bradford assay (Pierce Biotechnology). Protein was separated by sodium dodecylsulphate-polyacrylamide gel electrophoresis (SDS-PAGE) on 8, 10, or 16% resolving gels (Life Technologies). Proteins were then transferred to PVDF membrane (Millipore). Imaging was performed using a GE ImageQuant LAS 4000 imaging station. Quantification was performed by densitometry using ImageJ software.

### Results.

Our previous reports suggest that compound 390 is an mTORC1-selective inhibitor that does not inhibit mTORC2, unlike rapamycin which inhibits both mTORC1 and mTORC2 activities. Because inhibition of mTORC2 is thought to be responsible for several of the adverse effects of rapamycin, such as increased body weight, impaired insulin sensitivity, glucose homeostasis, we tested if compound 390 treatment would not lead to these metabolic side effects.

Glycemic Control.

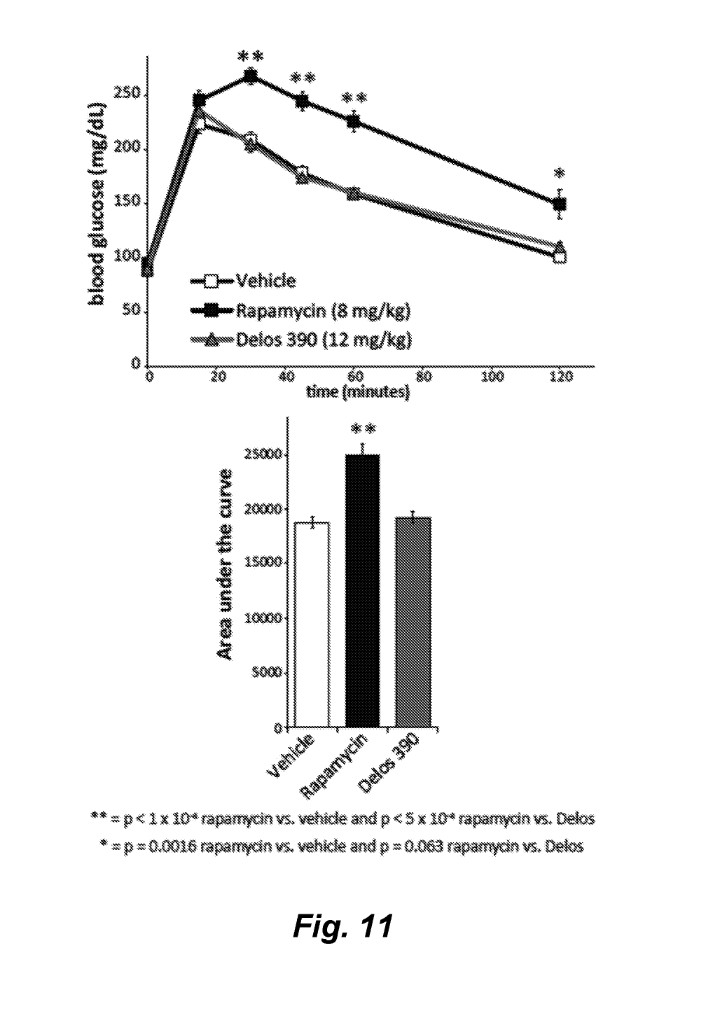

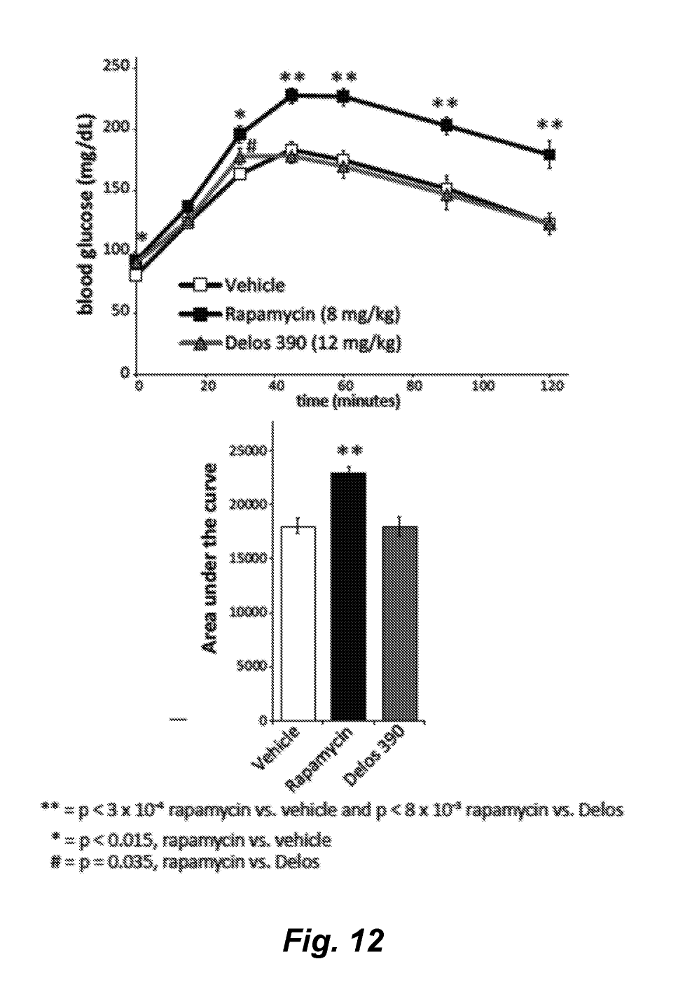

As indicated in FIGS. 11 and 12, chronic rapamycin treatment led to impaired glucose and pyruvate tolerance tests, as indicated by the substantially elevated glucose levels following both these challenges. In contrast, chronic compound 390 administration, even at 50% higher dose levels, produced glucose responses that were identical to the vehicle-receiving animals for both tests.

Adipocity.

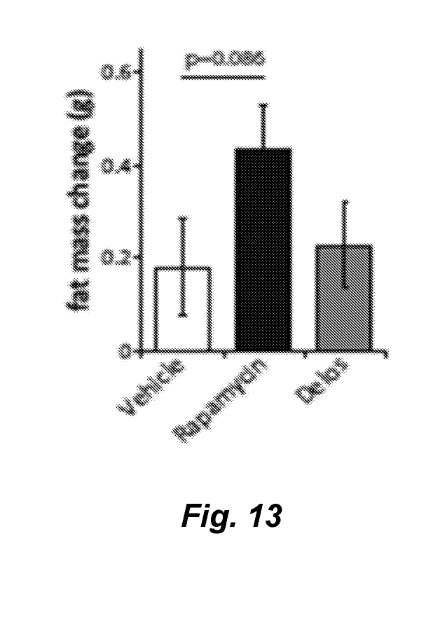

Following 4 weeks of every other day administration, rapamycin (N=18) produces a statistically significant increase of fat mass in the muscle tissue compared to vehicle controls. In contrast, compound 390 does not induce any increase of adiposity (see, e.g., FIG. 13).

mTORC1/2 Inhibition.

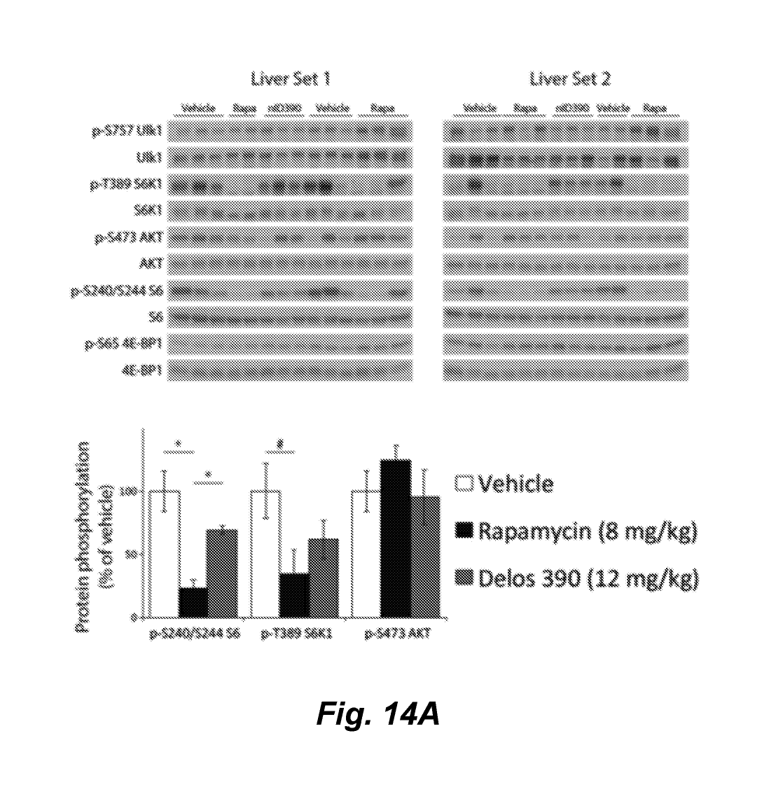

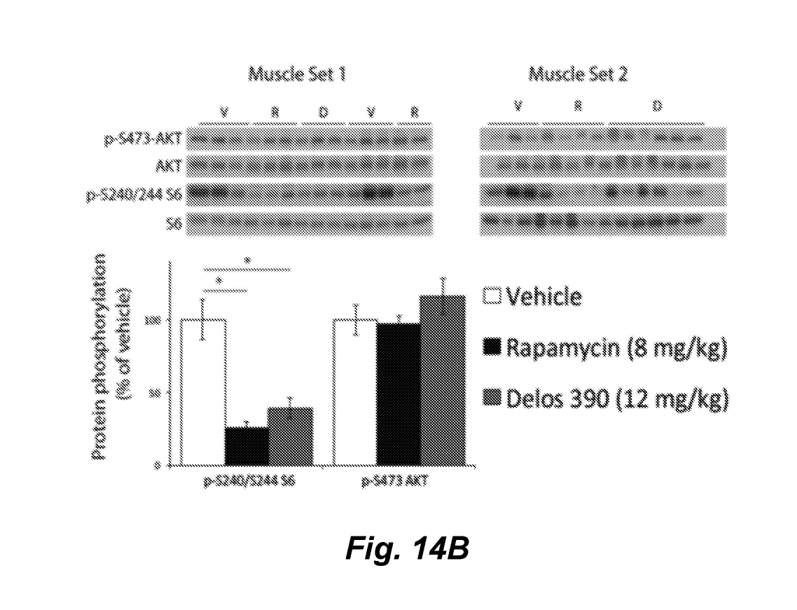

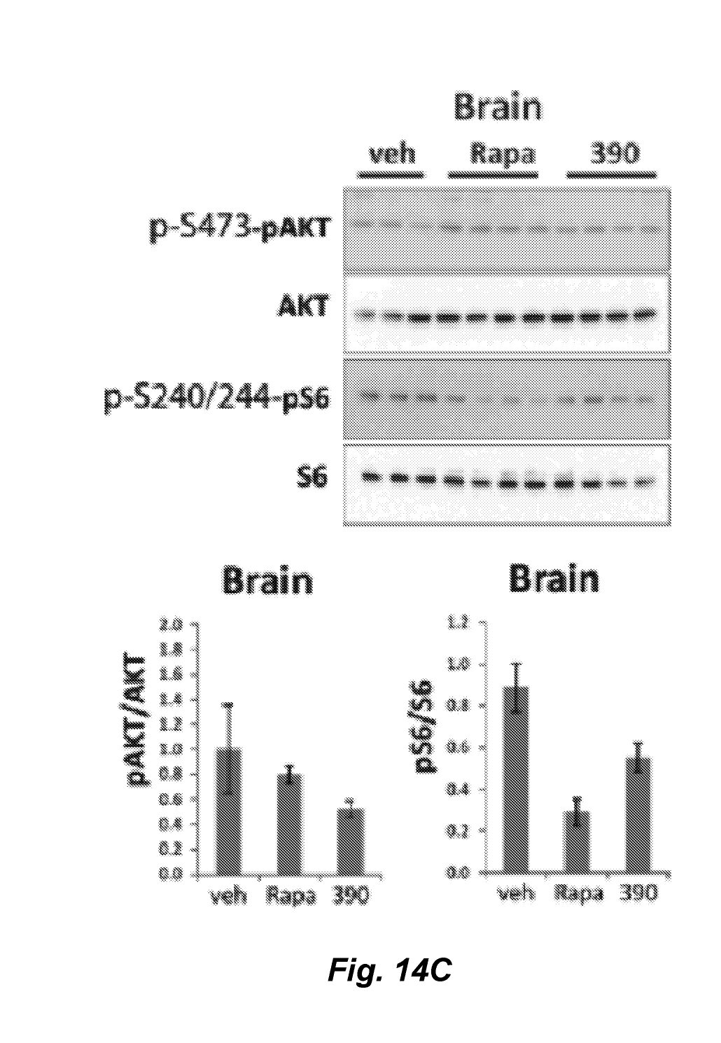

As shown in FIGS. 14A-14C, compound 390 significantly inhibits mTORC1 at approximately the same level as rapamycin in the liver, muscle and brain; interestingly in these tissues, both compounds appear to exhibit similar extend of mTORC2 inhibition.

mTORC2 Assembly Assessment.

It has been established that prolonged rapamycin treatment results in physical disassociation of the mTORC2 complex that can be detected by immunoprecipitation of the complex (see, e.g., Lamming et al. (2012) Science, 335(6076): 1638-1643).

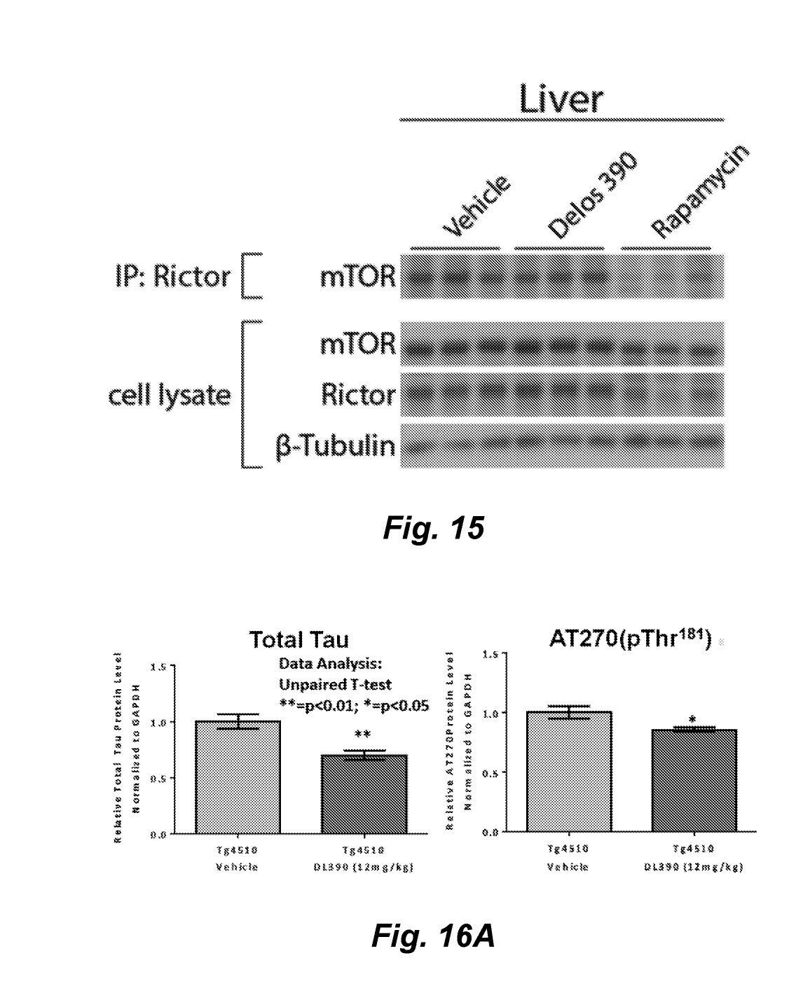

In order to determine the impact of both compounds (rapamycin and compound 390) on the state of mTORC2, we determined the physical integrity of mTORC2 by immunopreciptating Rictor from the liver and immunoblotting for mTOR. As shown in FIG. 15, rapamycin significantly disrupts the integrity of mTORC2 complex. In contrast, and consistent with our physiological data, compound 390 had no effect upon hepatic mTORC2 assembly.

Our analysis thus far suggests that compound 390 inhibits mTORC1 in vivo in multiple tissues in mice without disrupting mTORC2. Treatment of mice with compound 390 does not cause glucose intolerance, pyruvate intolerance, or fasting hyperglycemia, all of which are induced by rapamycin.

### Example 7

### In Vivo Assessment of the Efficacy of Compound 390, an mTORC1-Selective Inhibitor on Tg4510 Mutant Mice, a Model of Taupathy

### Purpose.

The objectives of this study were to assess:
i) the impact of compound 390 on the tau pathology and its efficacy in disease therapy;
ii) its mechanism of action and specifically its effects on the autophagy mechanisms in the brain of Tg4510 mice; and
iii) the cognitive-enhancing efficacy of compound 390

### Methods.

Female Tg4510 and control mice were purchased from the Jackson Laboratory (Bar Harbor, Me.). All mice were examined and weighed prior to initiation of the study to ensure adequate health and suitability and were caged under controlled temperature (20-23° C.) and relative humidity (maintained around 50%). All tests were performed during the animal's light cycle phase. Mice received every-other-day injections of either vehicle or compound 390 (12 mg/kg) in the following groups: (a) wild-type (WT) mice receiving vehicle (n=16); (b) Tg4510 mice receiving vehicle (n=15) and (c) Tg4510 mice receiving DL390 (12 mg/kg; n=15)

Morris Water Maze Test.

The test was performed in a circular tank, measuring 48″ in diameter. Extra-maze cues were mounted around the water tank. On 5 consecutive days of the week, test sessions were conducted with the platform submerged approximately 1.3 cm below the surface of the water. On each day, there were 4 trials, 60 s long. Approximately 15 min elapsed between each trial. On the fifth day of acquisition training, the last trial (out of 4) consisted of 60 s without the platform.

Tau Pathology Assessment.

Upon completion of the perfusion procedures, the hippocampus and cortex was dissected and frozen on dry ice and stored at −80° C. until analyses. The soluble and insoluble fractions of Tg4510 and WT mice (n=8 per group) brain were prepared by homogenization; the homogenates were centrifuged at 15,000 g for 15 min to remove the tissue debris. The supernatants were re-suspended after a second centrifugation at 100,000 g for 30 min. The pellets and supernatants from the second spin are defined as the insoluble and soluble fractions, respectively. Protein concentrations were determined using BioRad's DC Assay Kit. Total Tau Ab (Millipore), phosphorylated Tau (pTau) antibodies pTau AT270 (pThr181), pTau AT8 (pSer202/pThr205) and pTau AT180 (pThr231) (Life Technologies) were used to determine the total Tau and pTau levels in the soluble and insoluble fractions.

Evaluation of Autophagy Markers.

A number of autophagy markers were measured both in the cortex and hippocampus tissue to assess the status of the autophagic clearance mechanism across the three study groups. Specifically, pULK/ULK, Beclin-1, Vps34, LC3B and p62 were measured using the appropriate antibodies (Cell Signaling Technology).

Data were analyzed by analysis of variance (ANOVA) followed by post-hoc comparisons where appropriate. An effect was considered significant if p<0.05. All data are represented as the mean and standard error to the mean (s.e.m).

### Results.

Tau Pathology Efficacy.

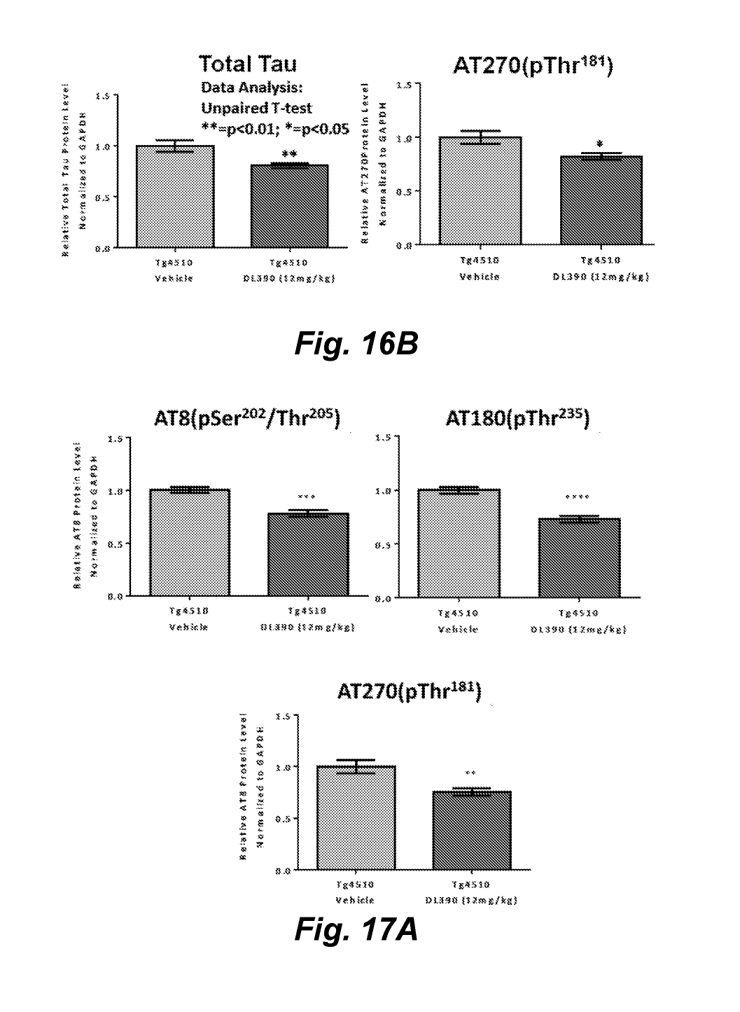

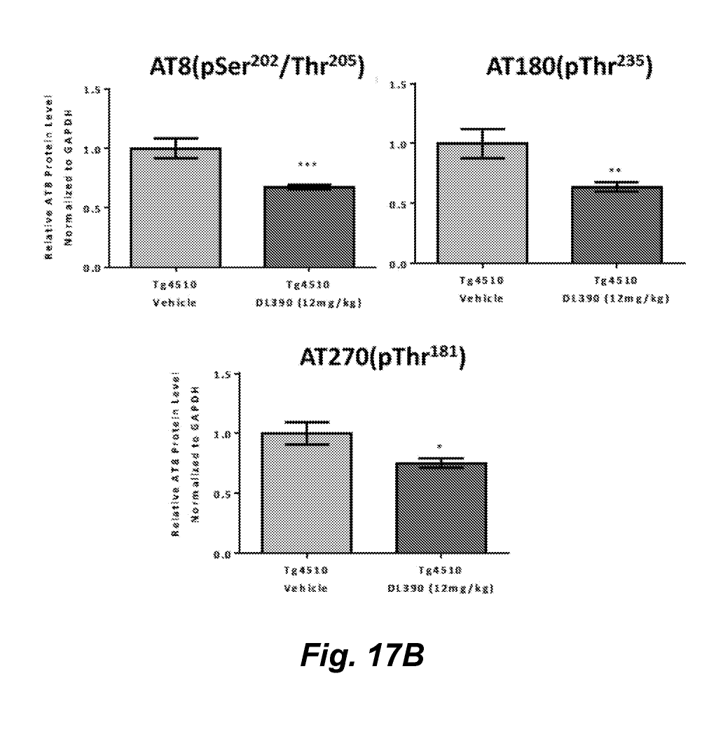

As shown in FIGS. 16A and 16B, even after a moderate (for efficacy) exposure of 4 weeks, administration of compound 390 results in substantial and significant reductions of both total Tau and p-Tau in the soluble fraction of both the cortex and the hippocampus. In addition, compound 390 induces a very substantial and statistically significant reduction of the levels of p-Tau in the insoluble fraction (FIGS. 17A and 17B) as determined by al three distinct antibodies. These results are indicative of the potency of the mTORC1 pathway in reducing the disease pathology. Compound 390's inhibitory action on mTORC1 results in substantial reduction of the levels of aberrant Tau proteins both in the soluble and insoluble fraction in the brain, while further confirms the brain permeability of the compound.

Autophagy Regulation Performance.

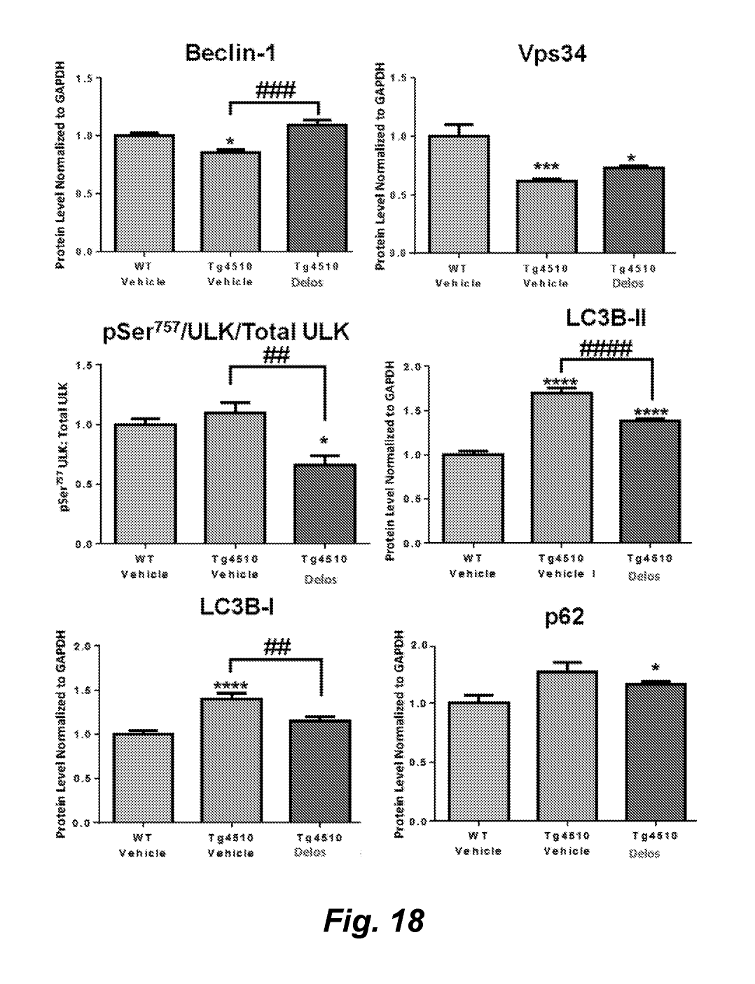

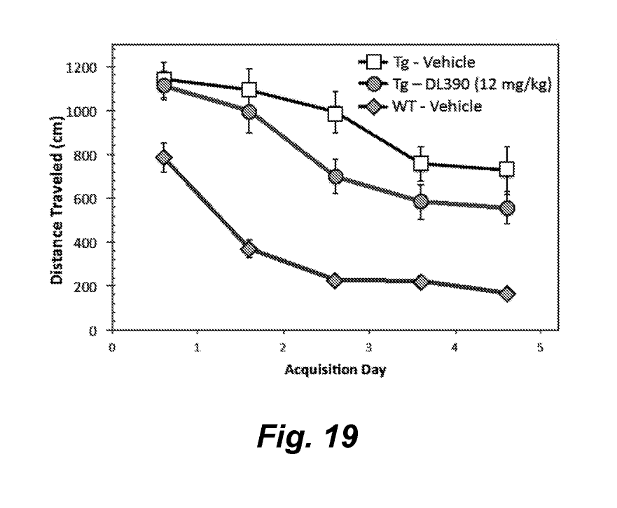

The results shown in FIG. 18 indicate the significant effects of mTORC1 inhibition, as enabled by compound 390, on the autophagic cascade. Compound 390 enables substantial and statistically significant upregulation of all stages of autophagy in the brain as indicated by the reduction of ULK1 phosphorylation (induction phase), nearly full restoration of Beclin-1 activity (initiation phase), substantial reduction of the accumulated LC3B-II and p62 levels (maturation and lysosomal clearance).

Behavioral Performance.

The total distance travelled during acquisition days are shown in FIG. 6.4. The overall ANOVA revealed a significant Treatment effect, days effect and treatment×day interaction. Post hoc analysis showed that during all acquisition days, vehicle-treated Tg4510 mice travelling greater distance before finding the platform compared to vehicle-treated WT mice and also compared to compound 390 (DL390) (12 mg/kg) treated Tg4510 mice on day 3 only. The behavioral effects do not reach statistical significance, which would not be expected after a short 4-weeks treatment period. However, they provide strong trends of behavioral improvement.

### Example 8

### Isolation of 27-O-Desmethyl-39-Desmethoxy Rapamycin (Compound 390)

Methods of production and isolation were carried out as generally as described in WO04/007709. The S. rapamycinicus strain MG2-10 (see WO04/007709) was conjugated with pLSS227, containing rapJ, rapM, rapN, rapO and rapLhis in pSGSet1. This plasmid was constructed as described in PCT Publication WO/04/007709. The strain was grown as follows:

### Fermentation of 27-O-Desmethyl-39-Desmethoxy Rapamycin

TABLE 6
RapV7 seed medium recipe.

| Ingredient | | Manufacturer | Quantity | |
|---|---|---|---|---|
| Corn steep solids | | | 4.0 | g |
| Soy flour (Toasted Nutrisoy) | | ADM | 5.0 | g |
| White dextrin WBD | | Avedex | 35.0 | g |
| Ammonium sulphate | | Reidel de Hahn | 2.0 | g |
| Lactic acid | | Fluka | 1.6 | ml |
| Calcium carbonate | | Sigma | 7 | g |
| Tap H₂O | to final vol. of | | 1.0 | L |

Adjust to pH 7 with 1 M NaOH.

Sterilize by heating 121° C., 30 min (autoclaving).

10 g/L (10 ml/L 40% solution) filter sterilized D-glucose added post sterilization.

### Seed Culture Preparation.

2000 ml Erlenmeyer flasks were filled with 400 ml RapV7 seed medium and sterilized by autoclave (121° C.; 30 min). A frozen (−80° C.) spore stock of S. rapamycinicus MG2-10 (pLSS227) was fully thawed and a 0.05% inoculum added to 400 ml sterile RapV7 seed medium which was pre-warmed and oxygenated at 28° C., 250 rpm, 2.5 cm throw for 30 min. This was incubated at 28° C., 250 rpm, 2.5 cm throw for 48 hrs.

### Production Medium Recipe

TABLE 7
MD6 production medium.

| Ingredient | Manufacturer | Quantity | |
|---|---|---|---|
| Soy flour (Toasted Nutrisoy) | ADM | 30 | g |
| Corn Starch | Sigma | 30 | g |
| White dextrin WBD | Avedex | 19 | g |
| Whole yeast | Fermipan | 3 | g |
| Corn steep solids | Roquette | 1 | g |
| K₂HPO₄ | Sigma | 2.5 | g |
| KH₂PO₄ | Sigma | 2.5 | g |
| Ammonium sulphate | Reidel de Haen | 10 | g |
| Sodium chloride | Fisher | 5 | g |
| Calcium carbonate | Sigma | 10 | g |
| MnCl₂•4H₂O | | 0.01 | g |
| MgSO₄•7H₂O | | 0.0025 | g |
| FeSO₄•7H₂O | Sigma | 0.12 | g |
| ZnSO₄•7H₂O | | 0.05 | g |
| MES | Acros Organics | 21.2 | g |
| α-Amylase | Sigma | 0.4 | ml |
| SAG471 (antifoam) | GE | 0.5 | ml |

| | |
|---|---|
| Tap water | to 1 L |

pH should be 6.0-7.0. Adjust prior to sterilization, if necessary.

Sterilization by autoclave (121° C.; 30 min).

TABLE 8
Post-sterilization additions to MD6 base medium (filter sterilized).

| Ingredient | Quantity | |
|---|---|---|
| 40% D-fructose | 50 | ml/L |
| 10% L-lysine (monohydrochloride) | 20 | ml/L |

Trans-4-hydroxy cyclohexane carboxylic acid was prepared 24 hours in advance in MeOH; final concentration 2 mM. 15 liters base medium (MD6 production medium without fructose or L-lysine) was transferred to a V7 Braun 22 L fermenter and sterilized. Following autoclaving, pre-sterilized fructose (15 g/L) and L-lysine (0.5 g/L) were added. The entire seed culture (400 ml) was transferred to production media in the fermentation vessel. S tarting parameters were T=26° C., 7.5 L/min air, 200 rpm, Aeration rate was: 0.5 v/v/m, automatic pH control to pH set point 6.5 (6.4-6.6), pH controlled with 15% NaOH. Dissolved oxygen was controlled with agitation cascade at 30% air saturation. Trans-4-hydroxy cyclohexane carboxylic acid in MeOH was added at 24 hours of fermentation to final concentration of 2 mM. SAG 471 (0.5 ml/L) used to prevent extensive foaming. The bioprocess was continued for 6 days.

### Preparation of Crude Extract of 27-O-Desmethyl-39-Desmethoxy Rapamycin

The whole broth was centrifuged at 3500 rpm (RCF 3300 g), 25 min. Clarified broth was assayed and discarded if less than 5% target compound detected. Cell pellet was removed from centrifuge pots with acetonitrile and decanted into 10 L duran. Further acetonitrile was added to give solvent to cell volume ration of 2:1; mixture stirred with overhead electric paddle stirrer, 600 rpm, 1 hour. Following stirring, the mixture was left to settle under gravity for 15 min. The solvent/aqueous layer was removed as extract 1. A further 2 volumes of acetonitrile were added to remaining cells; the mixture stirred and allowed to settle again, as above, to obtain extract 2. Any remaining 27-O-desmethyl-39-desmethoxy rapamycin in cell pellet was removed by third extraction, if required.

Extracts from cell biomass were concentrated in vacuo to residual aqueous extract. The aqueous fraction was extracted into an equal volume of ethyl acetate. The Ethyl acetate extract was concentrated in vacuo to yield an oily crude extract. This was dissolved in 80% MeOH in water and mixed with 1 volume hexane. The hexane partition was discarded and solvent removed in vacuo to yield final crude extract.

### Silica Chromatography

The crude extract was dissolved in methanol and a quantity of silica gel approximately equal to that of the extract added. Solvent was removed in vacuo to yield a free-flowing powder. Impregnated silica was loaded onto a silica gel column (20×5 cm) and eluted with 100% CHCl₃. Polarity was gradually increased by addition of MeOH to maximum of 5% MeOH. Approximately 20×250 ml fractions were collected and monitored by HPLC. Fractions containing any remaining 27-O-desmethyl-39-desmethoxy rapamycin were loaded onto second silica gel column (15×2 cm) and eluted with gradient of hexane and ethyl acetate, starting with 1 L 1:1 mixture, followed by 1 L 40:60 mixture and finally with 100% EtOAc. Approximately 20×250 ml fractions were collected and monitored by HPLC. Fractions containing any remaining 27-O-desmethyl-39-desmethoxy rapamycin were combined and the solvents removed in vacuo to yield semi pure compound.

### Final Purification by Preparative HPLC

Preparative HPLC was then performed using Waters X-Terra MS C18 column (OBD 10 μm; 19×250 mm) with security guard. The extract was dissolved in acetonitrile and 10 injections loaded onto column. Elution was in 55% to 80% acetonitrile in water gradient for 30 min. Fractions containing pure Any remaining 27-O-desmethyl-39-desmethoxy rapamycin were pooled and solvent removed in vacuo.

### Analysis of 27-O-Desmethyl-39-Desmethoxy Rapamycin by HPLC

Detection of 27-O-desmethyl-39-desmethoxy rapamycin was carried out using a Phenomenex Gemini-NX C18 3u 110A reversed-phase column (150×4.6 mm, 3 μm particle size) with security guard cartridge containing same silica as column.

System 1:

Mobile phase A: water:acetonitrile (9:1) containing 0.01 M ammonium acetate and 0.1% TFA. Mobile phase B: water acetonitrile (1:9) containing 0.01 M ammonium acetate and 0.1% TFA; RT 9.7 min.

System 2:

Mobile phase A: water+0.1% formic acid. Mobile phase B: acetonitrile+0.1% formic acid; RT 8.2 min.

Gradient:

Flow rate: 1 ml/min. Column oven temperature: 5° C. λₘₐₓ: 280 nm. Gradient: T=0 min, 55% B; T=10 min, 95% B; T=12 min, 95% B; T=12.5 min, 55% B; T=15 min, 55 min.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound of formula (I): 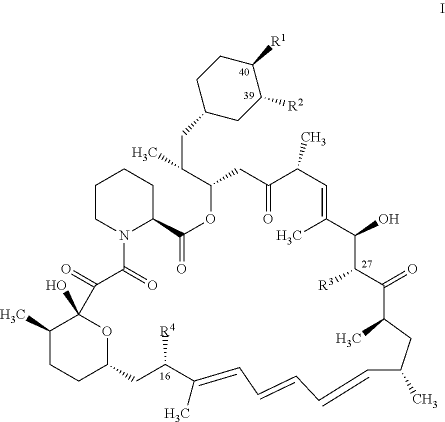
or a pharmaceutically acceptable salt thereof, wherein:
R¹ is OH or OCH₃
R² is H or F
R³ is H, OH, or OCH₃; and
R⁴ is OH or OCH₃.

2. The compound of claim 1, wherein said compound is in pure chiral form as a single diastereomer of formula II:

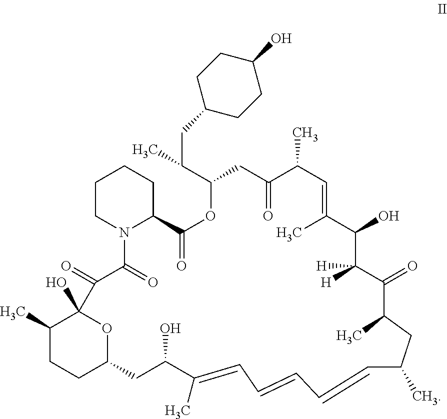

3. The compound of claim 1, wherein said compound is in pure chiral form as a single diastereomer of formula III:

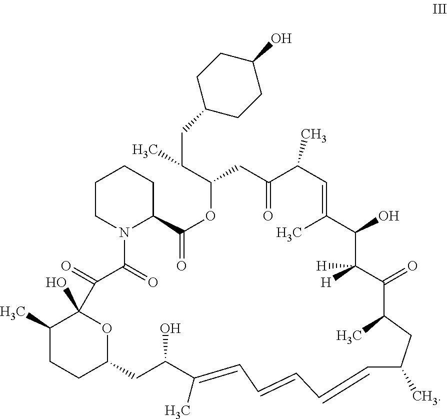

4. The compound of claim 1, wherein said compound is in substantially pure chiral form as a single diastereomer of formula IV:

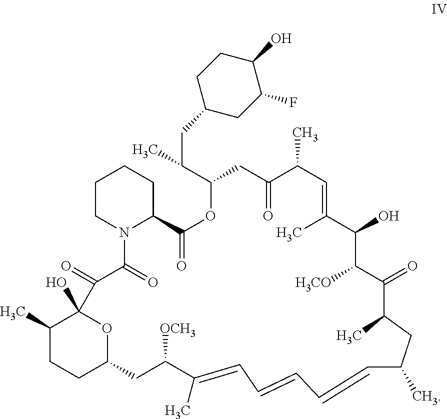 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](F)C2)CC(=O)[C@H](C)/C=C(\C)[C@@H](O)[C@@H](OC)C(=O)[C@H](C)C[C@H](C)/C=C/C=C/C=C/1C]

5. The compound of claim 1, wherein said compound is in substantially pure chiral form as a single diastereomer of formula V:

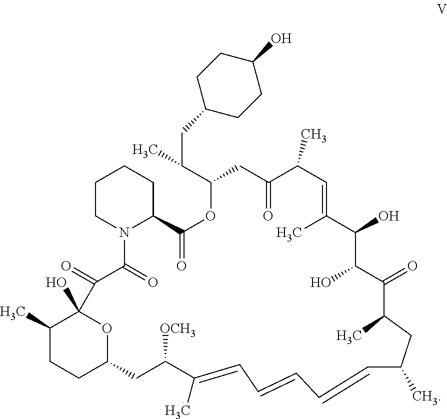 [SMILES: CO[C@H]1C[C@@H]2CC[C@@H](C)[C@@](O)(O2)C(=O)C(=O)N2CCCC[C@H]2C(=O)O[C@H]([C@H](C)C[C@H]2CC[C@H](O)CC2)CC(=O)[C@H](C)/C=C(\C)[C@@H](O)[C@@H](O)C(=O)[C@H](C)C[C@H](C)/C=C/C=C/C=C/1C]

6. The compound of claim 1, wherein said compound is in substantially pure chiral form as a single diastereomer of formula VI:

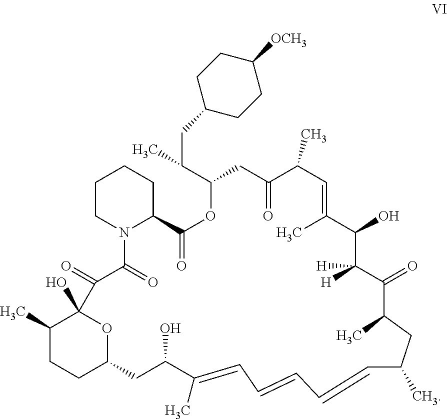

7. The compound according to any one of claims 1-6, wherein said compound is a preferential mTORC1 inhibitor.

8. A pharmaceutical formulation comprising:
a compound according to any one of claims 1-7; and
a pharmaceutically acceptable carrier or excipient.

9. The formulation of claim 8, wherein said formulation is a unit dosage formulation.

10. The formulation according to any one of claims 8-9, wherein said formulation is sterile.

11. The formulation according to any one of claims 8-10, wherein said formulation is formulated for administration via a route selected from the group consisting of administration via inhalation, aerosol administration, intravenous administration, intraarterial administration, oral administration, parenteral delivery, rectal administration, subdural administration, systemic administration, topical administration, transdermal delivery, and vaginal administration.

12. A compound according to any one of claims 1-7, or a pharmaceutical formulation according to any one of claims 8-11 for use in one or more of the following: the treatment of a tauopathy, the treatment of an mTORopathy, the treatment of an mTORopathy associated with epileptic seizures, the treatment of familial multiple discoid fibromas (FMDF), the treatment of an epilepsy/epileptic seizures (both genetic and acquired forms of the disease such as familial focal epilepsies, epileptic spasms, infantile spasms (IS), status epilepticus (SE), temporal lobe epilepsy (PLE) and absence epilepsy), the treatment of rare diseases associated with a dysfunction of mTORC1 activity, the treatment of the treatment of metabolic diseases, the treatment of autoimmune and inflammatory diseases, the treatment of cancer, the treatment of a fungal infection, the treatment of a proliferative disease, the maintenance of immunosuppression, the treatment of transplant rejection, the treatment of traumatic brain injury, the treatment of autism, the treatment of lysosomal storage diseases and the treatment of neurodegenerative diseases associated with an mTORC1 hyperactivity, and treatment of disorders that result in hyperactivation of the mTORC1 pathway.

13. The compound or pharmaceutical formulation of claim 12, for use in the treatment of a tauopathy.

14. The compound or pharmaceutical formulation of claim 13, for use in the treatment of a tauopathy selected from the group consisting of progressive supranuclear palsy, dementia pugilistica (chronic traumatic encephalopathy), frontotemporal dementia, lytico-bodig disease (parkinson-dementia complex of guam), tangle-predominant dementia (with nfts similar to ad, but without plaques), ganglioglioma and gangliocytoma, meningioangiomatosis, subacute sclerosing panencephalitis, lead encephalopathy, tuberous sclerosis, Pick's disease, corticobasal degeneration (tau proteins are deposited in the form of inclusion bodies within swollen or “ballooned” neurons), Alzheimer's disease, Parkinson's disease, Huntington's disease, frontotemporal dementia, frontotemporal lobar degeneration.

15. The compound or pharmaceutical formulation of claim 12, for use in the treatment of an mTORpathy.

16. The compound or pharmaceutical formulation of claim 15, wherein said mTORpathy comprises a pathology selected from the group consisting tuberous sclerosis complex (TSC), focal cortical dysplasia (FCD), ganglioglioma, hemimegalencephaly, neurofibromatosis 1, Sturge-Weber syndrome, Cowden syndrome, and PMSE (Polyhydramnios, Megalencephaly, Symptomatic Epilepsy)).

17. The compound or pharmaceutical formulation of claim 12, for use in the treatment of a pathology selected from the group consisting of epilepsy, neurodegeneration, rare and genetic disease with mTORC1 hyperactivity, metabolic disease, and traumatic brain injury.

18. The compound or pharmaceutical formulation of claim 12, for use in the treatment of cancer.

19. The compound or pharmaceutical formulation of claim 18, wherein said cancer is a cancer selected from the group consisting of acute lymphoblastic leukemia (ALL), acute myeloid leukemia (AML), Adrenocortical carcinoma, kaposi sarcoma, anal cancer, appendix cancer, astrocytomas, atypical teratoid/rhabdoid tumor, bile duct cancer, extrahepatic cancer, bladder cancer, bone cancer, brain stem glioma, astrocytomas, spinal cord tumors, central nervous system atypical teratoid/rhabdoid tumor, central nervous system embryonal tumors, central nervous system germ cell tumors, craniopharyngioma, ependymoma, breast cancer, bronchial tumors, burkitt lymphoma, carcinoid tumors, cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, bile duct cancer, extrahepatic cancer, ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, intraocular melanoma, retinoblastoma, fibrous histiocytoma of bone, malignant, and osteosarcoma, gallbladder cancer, gastric (stomach) cancer, gastrointestinal carcinoid tumor, gastrointestinal stromal tumors (GIST), ovarian cancer, testicular cancer, extracranial cancers, extragonadal cancers, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kidney cancer, langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer, lymphoma, cutaneous T-Cell cancer, Hodgkin, non-Hodgkin, primary central nervous system (CNS)), macroglobulinemia, Waldenström, male breast cancer, melanoma, merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma, skin cancer, small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

20. The compound or pharmaceutical formulation of claim 18, wherein said cancer is a cancer selected from the group consisting of brain cancer, breast cancer, central nervous system cancer, cervical cancer, colorectal cancer, testicular cancer, ovarian cancer, leukemia, a lymphoma, a melanoma, a soft tissue sarcoma, testicular cancer, and thyroid cancer.

21. The compound or pharmaceutical formulation of claim 12, for use in the prevention of transplant rejection.

22. The compound or pharmaceutical formulation of claim 21, for use in combination with a calcineurin inhibitor and/or glucocorticoid for the prevention of transplant rejection.

23. The compound or pharmaceutical formulation of claim 21, for use in combination with cyclosporine for the prevention of transplant rejection.

24. The compound or pharmaceutical formulation of claim 12, for use in the treatment of an autoimmune disease.

25. The compound or pharmaceutical formulation of claim 24, wherein said autoimmune disease comprises lupus.

26. The compound or pharmaceutical formulation of claim 24, wherein said autoimmune disease comprises multiple sclerosis.

27. The compound or pharmaceutical formulation of claim 12, for use in the treatment of an infection, autism, or a lysosomal storage disease.

28. A method of preparing a compound according to any one of claims 1, 2, or 3, said method comprising providing the feed starter (1R,4R)-4-hydroxycyclohexanecarboxylic acid in pure chiral form of formula (VII) 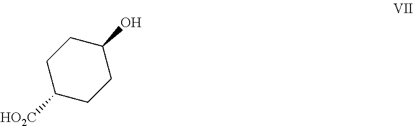
to a rapamycin producing strain of Streptomyces rapamycinicus that has been genetically altered to delete the genes rapI, rapJ, rapK, rapL, rapM, rapN, rapO, and rapQ and conjugated with a plasmid containing rapJ, rapM, rapN, rapO and rapLhis.

29. A method of preparing a compound according to any one of claims 1, 5, or 6, said method comprising providing the feed starter (1R,4R)-4-methoxycyclohexanecarboxylic acid in pure chiral form of formula (VIII) 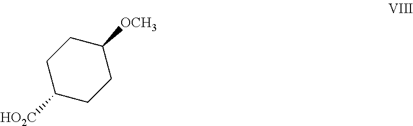 [SMILES: CO[C@H]1CC[C@H](C(=O)O)CC1]
to a rapamycin producing strain of Streptomyces rapamycinicus that has been genetically altered to delete the genes rapI, rapJ, rapK, rapL, rapM, rapN, rapO, and rapQ and conjugated with a plasmid containing rapJ, rapM, rapN, rapO and rapLhis.

30. A method of preparing a compound according to any one of claims 1 or 4, said method comprising providing the feed starter (1R,3R,4R)-3-fluoro-4-hydroxycyclohexane carcarboxylic acid in pure chiral form of formula (IX) 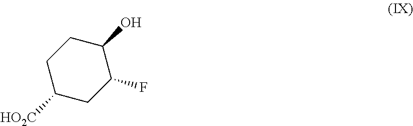 [SMILES: O=C(O)[C@@H]1CC[C@@H](O)[C@H](F)C1]
to a rapamycin producing strain of Streptomyces rapamycinicus that has been genetically altered to delete the genes rapI, rapJ, rapK, rapL, rapM, rapN, rapO, and rapQ and conjugated with a plasmid containing rapJ, rapM, rapN, rapO and rapLhis.

31. The method according to any one of claims 28-30, wherein said strain is S. rapamycinicus strain MG2-10.

32. A compound according to the formula: 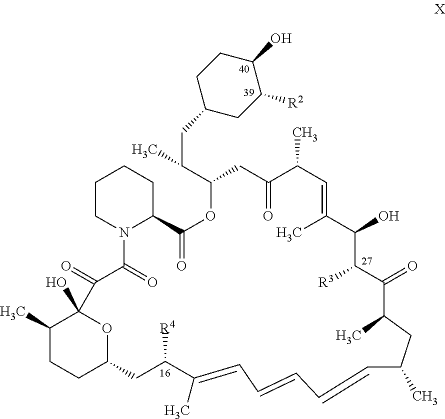
or a pharmaceutically acceptable salt thereof, wherein:
R² is H or F;
R³ is OH, or OCH₃; and
R⁴ is OCH3 or OH.

33. The compound of claim 32, wherein R⁴ is OCH₃.

34. The compound of claim 33, wherein R² is F and R³ is OCH₃.

35. The compound of claim 33, wherein R² is H, and R³ is OH.

36. The compound of claim 32, wherein R² is H, R³ is H, and R⁴ is OH.

37. A pharmaceutical formulation comprising:
a compound according to any one of claims 32-36; and
a pharmaceutically acceptable carrier or excipient.

38. The formulation of claim 37, wherein said formulation is a unit dosage formulation.

39. The formulation according to any one of claims 37-38, wherein said formulation is sterile.

40. The formulation according to any one of claims 37-39, wherein said formulation is formulated for administration via a route selected from the group consisting of administration via inhalation, aerosol administration, intravenous administration, intraarterial administration, oral administration, parenteral delivery, rectal administration, subdural administration, systemic administration, topical administration, transdermal delivery, and vaginal administration.

41. A compound according to any one of claims 32-36, or a pharmaceutical formulation according to any one of claims 37-40 for use in one or more of the following: the treatment of a tauopathy, the treatment of an mTORopathy, the treatment of an mTORopathy associated with epileptic seizures, the treatment of familial multiple discoid fibromas (FMDF), the treatment of an epilepsy/epileptic seizures (both genetic and acquired forms of the disease such as familial focal epilepsies, epileptic spasms, infantile spasms (IS), status epilepticus (SE), temporal lobe epilepsy (PLE) and absence epilepsy), the treatment of rare diseases associated with a dysfunction of mTORC1 activity, the treatment of the treatment of metabolic diseases, the treatment of autoimmune and inflammatory diseases, the treatment of cancer, the treatment of a fungal infection, the treatment of a proliferative disease, the maintenance of immunosuppression, the treatment of transplant rejection, the treatment of traumatic brain injury, the treatment of autism, the treatment of lysosomal storage diseases and the treatment of neurodegenerative diseases associated with an mTORC1 hyperactivity, and treatment of disorders that result in hyperactivation of the mTORC1 pathway.

42. The compound or pharmaceutical formulation of claim 41, for use in the treatment of a tauopathy.

43. The compound or pharmaceutical formulation of claim 42, for use in the treatment of a tauopathy selected from the group consisting of progressive supranuclear palsy, dementia pugilistica (chronic traumatic encephalopathy), frontotemporal dementia, lytico-bodig disease (parkinson-dementia complex of guam), tangle-predominant dementia (with nfts similar to ad, but without plaques), ganglioglioma and gangliocytoma, meningioangiomatosis, subacute sclerosing panencephalitis, lead encephalopathy, tuberous sclerosis, Pick's disease, corticobasal degeneration (tau proteins are deposited in the form of inclusion bodies within swollen or “ballooned” neurons), Alzheimer's disease, Parkinson's disease, Huntington's disease, frontotemporal dementia, frontotemporal lobar degeneration.

44. The compound or pharmaceutical formulation of claim 41, for use in the treatment of an mTORpathy.

45. The compound or pharmaceutical formulation of claim 44, wherein said mTORpathy comprises a pathology selected from the group consisting tuberous sclerosis complex (TSC), focal cortical dysplasia (FCD), ganglioglioma, hemimegalencephaly, neurofibromatosis 1, Sturge-Weber syndrome, Cowden syndrome, and PMSE (Polyhydramnios, Megalencephaly, Symptomatic Epilepsy)).

46. The compound or pharmaceutical formulation of claim 41, for use in the treatment of a pathology selected from the group consisting of epilepsy, neurodegeneration, rare and genetic disease with mTORC1 hyperactivity, metabolic disease, and traumatic brain injury.

47. The compound or pharmaceutical formulation of claim 41, for use in the treatment of cancer.

48. The compound or pharmaceutical formulation of claim 47, wherein said cancer is a cancer selected from the group consisting of acute lymphoblastic leukemia (ALL), acute myeloid leukemia (AML), Adrenocortical carcinoma, kaposi sarcoma, anal cancer, appendix cancer, astrocytomas, atypical teratoid/rhabdoid tumor, bile duct cancer, extrahepatic cancer, bladder cancer, bone cancer, brain stem glioma, astrocytomas, spinal cord tumors, central nervous system atypical teratoid/rhabdoid tumor, central nervous system embryonal tumors, central nervous system germ cell tumors, craniopharyngioma, ependymoma, breast cancer, bronchial tumors, burkitt lymphoma, carcinoid tumors, cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, bile duct cancer, extrahepatic cancer, ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, intraocular melanoma, retinoblastoma, fibrous histiocytoma of bone, malignant, and osteosarcoma, gallbladder cancer, gastric (stomach) cancer, gastrointestinal carcinoid tumor, gastrointestinal stromal tumors (GIST), ovarian cancer, testicular cancer, extracranial cancers, extragonadal cancers, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kidney cancer, langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer, lymphoma, cutaneous T-Cell cancer, Hodgkin, non-Hodgkin, primary central nervous system (CNS)), macroglobulinemia, Waldenström, male breast cancer, melanoma, merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma, skin cancer, small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

49. The compound or pharmaceutical formulation of claim 47, wherein said cancer is a cancer selected from the group consisting of brain cancer, breast cancer, central nervous system cancer, cervical cancer, colorectal cancer, testicular cancer, ovarian cancer, leukemia, a lymphoma, a melanoma, a soft tissue sarcoma, testicular cancer, and thyroid cancer.

50. The compound or pharmaceutical formulation of claim 41, for use in the prevention of transplant rejection.

51. The compound or pharmaceutical formulation of claim 50, for use in combination with a calcineurin inhibitor and/or glucocorticoid for the prevention of transplant rejection.

52. The compound or pharmaceutical formulation of claim 50, for use in combination with cyclosporine for the prevention of transplant rejection.

53. The compound or pharmaceutical formulation of claim 41, for use in the treatment of an autoimmune disease.

54. The compound or pharmaceutical formulation of claim 53, wherein said autoimmune disease comprises lupus.

55. The compound or pharmaceutical formulation of claim 53, wherein said autoimmune disease comprises multiple sclerosis.

56. The compound or pharmaceutical formulation of claim 41, for use in the treatment of an infection, autism, or a lysosomal storage disease.

57. The compound or pharmaceutical formulation according to any one of claims 41-56 for use in the treatment of a human.

58. The compound or pharmaceutical formulation according to any one of claims 41-56 for use in the treatment of a non-human mammal.
